SEQUENCE LISTING (33 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 3142 aa, Homo sapiens
MATLEKLMKAFESLKSFQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 3 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 4 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 5 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 6 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 8 - protein, 2386 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 9 - protein, 154 aa, Homo sapiens [sequence omitted]

SEQ ID NO: 10 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 11 - protein, 724 aa, Homo sapiens
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 12 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 13 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 14 - protein, 679 aa, Homo sapiens
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 15 - protein, 108 aa, Homo sapiens
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 16 - protein, 459 aa, Homo sapiens
MTAEEMKATESGAQSAPLPMEGVDISPKQDEGVLKVIKREGTGTEMPMIGDRVFVHYTGWLLDGTKFDSSLDRKDKFSFDLGKGEVIKAWDIAIATMKVGEVCHITCKPEYAYGSAGSPPKIPPNATLVFEVELFEFKGEDLTEEEDGGIIRRIQTRGEGYAKPNEGAIVEVALEGYYKDKLFDQRELRFEIGEGENLDLPYGLERAIQRMEKGEHSIVYLKPSYAFGSVGKEKFQIPPNAELKYELHLKSFEKAKESWEMNSEEKLEQSTIVKERGTVYFKEGKYKQALLQYKKIVSWLEYESSFSNEEAQKAQALRLASHLNLAMCHLKLQAFSAAIESCNKALELDSNNEKGLFRRGEAHLAVNDFELARADFQKVLQLYPNNKAAKTQLAVCQQRIRRQLAREKKLYANMFERLAEEENKAKAEASSGDHPTDTEMKEEQKSNTAGSQSQVETEA

SEQ ID NO: 17 - protein, 189 aa, Homo sapiens
MASKRALVILAKGAEEMETVIPVDVMRRAGIKVTVAGLAGKDPVQCSRDVVICPDASLEDAKKEGPYDVVVLPGGNLGAQNLSESAAVKEILKEQENRKGLIAAICAGPTALLAHEIGFGSKVTTHPLAKDKMMNGGHYTYSENRVEKDGLILTSRGPGTSFEFALAIVEALNGKEVAAQVKAPLVLKD

SEQ ID NO: 18 - protein, 163 aa, Homo sapiens
MADEEKLPPGWEKRMSRSSGRVYYFNHITNASQWERPSGNSSSGGKNGQGEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

SEQ ID NO: 19 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 20 - protein, 370 aa, Homo sapiens
MSHPSPQAKPSNPSNPRVFFDVDIGGERVGRIVLELFADIVPKTAENFRALCTGEKGIGHTTGKPLHFKGCPFHRIIKKFMIQGGDFSNQNGTGGESIYGEKFEDENFHYKHDREGLLSMANAGRNTNGSQFFITTVPTPHLDGKHVVFGQVIKGIGVARILENVEVKGEKPAKLCVIAECGELKEGDDGGIFPKDGSGDSHPDFPEDADIDLKDVDKILLITEDLKNIGNTFFKSQNWEMAIKKYAEVLRYVDSSKAVIETADRAKLQPIALSCVLNIGACKLKMSNWQGAIDSCLEALELDPSNTKALYRRAQGWQGLKEYDQALADLKKAQGIAPEDKAIQAELLKVKQKIKAQKDKEKAVYAKMFA

SEQ ID NO: 21 - protein, 247 aa, Homo sapiens
MVDREQLVQKARLAEQAERYDDMAAAMKNVTELNEPLSNEERNLLSVAYKNVVGARRSSWRVISSIEQKTSADGNEKKIEMVRAYREKIEKELEAVCQDVLSLLDNYLIKNCSETQYESKVFYLKMKGDYYRYLAEVATGEKRATVVESSEKAYSEAHEISKEHMQPTHPIRLGLALNYSVFYYEIQNAPEQACHLAKTAFDDAIAELDTLNEDSYKDSTLIMQLLRDNLTLWTSDQQDDDGGEGNN

SEQ ID NO: 22 - protein, 255 aa, Homo sapiens
MDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 23 - protein, 245 aa, Homo sapiens
MEKTELIQKAKLAEQAERYDDMATCMKAVTEQGAELSNEERNLLSVAYKNVVGGRRSAWRVISSIEQKTDTSDKKLQLIKDYREKVESELRSICTTVLELLDKYLIANATNPESKVFYLKMKGDYFRYLAEVACGDDRKQTIDNSQGAYQEAFDISKKEMQPTHPIRLGLALNFSVFYYEILNNPELACTLAKTAFDEAIAELDTLNEDSYKDSTLIMQLLRDNLTLWTSDSAGEECDAAEGAEN

SEQ ID NO: 24 - protein, 205 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 25 - protein, 216 aa, Homo sapiens
MGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNGLSFIETSALDSTNVEAAFQTILTEIYRIVSQKQMSDRRENDMSPSNNVVPIHVPPTTENKPKVQCCQNI

SEQ ID NO: 26 - protein, 340 aa, Homo sapiens
MGKDYYQTLGLARGASDEEIKRAYRRQALRYHPDKNKEPGAEEKFKEIAEAYDVLSDPRKREIFDRYGEEGLKGSGPSGGSGGGANGTSFSYTFHGDPHAMFAEFFGGRNPFDTFFGQRNGEEGMDIDDPFSGFPMGMGGFTNVNFGRSRSAQEPARKKQDPPVTHDLRVSLEEIYSGCTKKMKISHKRLNPDGKSIRNEDKILTIEVKKGWKEGTKITFPKEGDQTSNNIPADIVFVLKDKPHNIFKRDGSDVIYPARISLREALCGCTVNVPTLDGRTIPVVFKDVIRPGMRRKVPGEGLPLPKTPEKRGDLIIEFEVIFPERIPQTSRTVLEQVLPI

SEQ ID NO: 27 - protein, 198 aa, Homo sapiens
MADQRQRSLSTSGESLYHVLGLDKNATSDDIKKSYRKLALKYHPDKNPDNPEAADKFKEINNAHAILTDATKRNIYDKYGSLGLYVAEQFGEENVNTYFVLSSWWAKALFVFCGLLTCCYCCCCLCCCFNCCCGKCKPKAPEGEETEFYVSPEDLEAQLQSDEREATDTPIVIQPASATETTQLTADSHPSYHTDGFN

SEQ ID NO: 28 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 29 - protein, 999 aa, Homo sapiens
MADKVRRQRPRRRVCWALVAVLLADLLALSDTLAVMSVDLGSESMKVAIVKPGVPMEIVLNKESRRKTPVIVTLKENERFFGDSAASMAIKNPKATLRYFQHLLGKQADNPHVALYQARFPEHELTFDPQRQTVHFQISSQLQFSPEEVLGMVLNYSRSLAEDFAEQPIKDAVITVPVFFNQAERRAVLQAARMAGLKVLQLINDNTATALSYGVFRRKDINTTAQNIMFYDMGSGSTVCTIVTYQMVKTKEAGMQPQLQIRGVGFDRTLGGLEMELRLRERLAGLFNEQRKGQRAKDVRENPRAMAKLLREANRLKTVLSANADHMAQIEGLMDDVDFKAKVTRVEFEELCADLFERVPGPVQQALQSAEMSLDEIEQVILVGGATRVPRVQEVLLKAVGKEELGKNINADEAAAMGAVYQAAALSKAFKVKPFVVRDAVVYPILVEFTREVEEEPGIHSLKHNKRVLFSRMGPYPQRKVITFNRYSHDFNFHINYGDLGFLGPEDLRVFGSQNLTTVKLKGVGDSFKKYPDYESKGIKAHFNLDESGVLSLDRVESVFETLVEDSAEEESTLTKLGNTISSLFGGGTTPDAKENGTDTVQEEEESPAEGSKDEPGEQVELKEEAEAPVEDGSQPPPPEPKGDATPEGEKATEKENGDKSEAQKPSEKAEAGPEGVAPAPEGEKKQKPARKRRMVEEIGVELVVLDLPDLPEDKLAQSVQKLQDLTLRDLEKQEREKAANSLEAFIFETQDKLYQPEYQEVSTEEQREEISGKLSAASTWLEDEGVGATTVMLKEKLAELRKLCQGLFFRVEERKKWPERLSALDNLLNHSSMFLKGARLIPEMDQIFTEVEMTTLEKVINETWAWKNATLAEQAKLPATEKPVLLSKDIEAKMMALDREVQYLLNKAKFTKPRPRPKDKNGTRAEPPLNASASDQGEKVIPPAGQTEDAEPISEPEKVETGSEPGDTEPLELGGPGAEPEQKEQSTGQKRPLKNDEL

SEQ ID NO: 30 - protein, 350 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVT

SEQ ID NO: 31 - protein, 858 aa, Homo sapiens
MSVVGLDVGSQSCYIAVARAGGIETIANEFSDRCTPSVISFGSKNRTIGVAAKNQQITHANNTVSNFKRFHGRAFNDPFIQKEKENLSYDLVPLKNGGVGIKVMYMGEEHLFSVEQITAMLLTKLKETAENSLKKPVTDCVISVPSFFTDAERRSVLDAAQIVGLNCLRLMNDMTAVALNYGIYKQDLPSLDEKPRIVVFVDMGHSAFQVSACAFNKGKLKVLGTAFDPFLGGKNFDEKLVEHFCAEFKTKYKLDAKSKIRALLRLYQECEKLKKLMSSNSTDLPLNIECFMNDKDVSGKMNRSQFEELCAELLQKIEVPLYSLLEQTHLKVEDVSAVEIVGGATRIPAVKERIAKFFGKDISTTLNADEAVARGCALQCAILSPAFKVREFSVTDAVPFPISLIWNHDSEDTEGVHEVFSRNHAAPFSKVLTFLRRGPFELEAFYSDPQGVPYPEAKIGRFVVQNVSAQKDGEKSRVKVKVRVNTHGIFTISTASMVEKVPTEENEMSSEADMECLNQRPPENPDTDKNVQQDNSEAGTQPQVQTDAQQTSQSPPSPELTSEENKIPDADKANEKKVDQPPEAKKPKIKVVNVELPIEANLVWQLGKDLLNMYIETEGKMIMQDKLEKERNDAKNAVEEYVYEFRDKLCGPYEKFICEQDHQNFLRLLTETEDWLYEEGEDQAKQAYVDKLEELMKIGTPVKVRFQEAEERPKMFEELGQRLQHYAKIAADFRNKDEKYNHIDESEMKKVEKSVNEVMEWMNNVMNAQAKKSLDQDPVVRAQEIKTKIKELNNTCEPVVTQPKPKIESPKLERTPNGPNIDKKEEDLEDKNNFGAEPPHQNGECYPNEKNSVNMDLD

SEQ ID NO: 32 - protein, 303 aa, Homo sapiens
MKGKEEKEGGARLGAGGGSPEKSPSAQELKEQGNRLFVGRKYPEAAACYGRAITRNPLVAVYYTNRALCYLKMQQHEQALADCRRALELDGQSVKAHFFLGQCQLEMESYDEAIANLQRAYSLAKEQRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 33 - protein, 173 aa, Homo sapiens
MDVTIQHPWFKRTLGPFYPSRLFDQFFGEGLFEYDLLPFLSSTISPYYRQSLFRTVLDSGISEVRSDRDKFVIFLDVKHFSPEDLTVKVQDDFVEIHGKHNERQDDHGYISREFHRRYRLPSNVDQSALSCSLSADGMLTFCGPKIQTGLDATHAERAIPVSREEKPTSAPSS

(19) United States
(12) Patent Application Publication
(10) Pub. No.: US 2016/0220649 A1
(43) Pub. Date: Aug. 4, 2016

(54) COMBINATIONS OF AGGREGATING PROTEINS AND MOLECULAR CHAPERONE PROTEINS FOR THE TREATMENT OF PROTEINOPATHIES OR CONFORMATIONAL DISEASES

(71) Applicants: FUNDACIÓN PÚBLICA ANDALUZA PROGRESO Y SALUD, Sevilla (ES); UNIVERSIDAD DE SEVILLA, Sevilla (ES)

(72) Inventors: Cintia Roodveldt Catellani, Sevilla (ES); Adahir Labrador Garrido, Sevilla (ES); David Pozo Pérez, Sevilla (ES)

(21) Appl. No.: 15/021,277

(22) PCT Filed: Sep. 15, 2014
(86) PCT No.: PCT/ES2014/070704

§ 371 Date: Mar. 11, 2016

(30) Foreign Application Priority Data
Sep. 13, 2013 (ES) P201331334

(51) Int. Cl. A61K 39/00; C07K 14/47; C07K 16/40; C07K 16/18

(52) U.S. Cl. CPC A61K 39/0007; C07K 16/18; C07K 14/47; C07K 16/40; A61K 2039/5154; A61K 2039/6043; C07K 2317/20; A61K 2039/575; A61K 2039/55566; A61K 2039/572; A61K 2039/57

(57) ABSTRACT

HSPs (chaperones or heat-shock proteins) have two very valuable characteristics that can be productively exploited in a specific manner in order to “intelligently” manipulate the immune response so as to treat “conformational” disorders or “proteinopathies”:
the classic chaperone functions thereof; and
the more recently discovered immunoactive properties thereof.

On the basis thereof, the authors of the present invention have combined a peptide, a protein or a polypeptide associated with a “conformational disease” with a biologically active or functional HSP, resulting in a different immunoactive complex which is advantageous for treating the “conformational disease” in question associated with the peptide, protein or polypeptide used.

The present invention is comprised within the field of medicine, molecular biology and pharmacy, and relates specifically to a vaccine comprising an aggregating peptide or polypeptide and a molecular chaperone in the preparation of a medicinal product or vaccine for the treatment and/or prevention of an aggregating protein-related disease. It also relates to the nucleotide sequences, expression vectors, host cells, antibodies or immunotherapeutic cellular composition related with the composition of the invention, as well as to a method for the diagnosis of aggregating protein-related diseases by means of using the antibodies of the invention.

### PRIOR STATE OF THE ART

Parkinson's disease (PD) is the second most common neurodegenerative disease after Alzheimer's disease, with a higher and increasing prevalence in industrialized countries. This disorder and other proteinopathies or conformational diseases are characterized by the poor folding and aggregation of specific proteins in highly ordered fibrillary structures that accumulate and give rise to the formation of protein deposits in certain cells and areas of the brain (Knowles T P et al. 2014. Nat Rev Mol Cell Biol. 15:384-96.). PD is pathologically characterized by the presence of α-synuclein (αSyn) aggregate deposits in intracellular inclusions known as Lewy bodies (LBs) in the substantia nigra of the brain and by the loss of dopamine-producing neurons. Despite the fact that the causes and the underlying pathogenic mechanism have not yet been completely clarified, it has been established that αSyn plays a fundamental role in the onset and progression of the disease (Marques & Outeiro, 2012. Cell Death Dis. 3: e350). Today, there is no effective treatment against this devastating disease the normal process of which ultimately leads to major loss of neuron cells and dementia.

The αSyn protein, the physiological functions of which are unknown today, is expressed fundamentally in neurons so it is mainly found in the brain. Despite the fact that αSyn has been conventionally considered as an exclusively intracellular protein, evidence showing the presence of αSyn, both in the aggregated and non-aggregated form, in the cerebrospinal fluid (CSF) and blood has been amassed. In fact, it has been found that disequilibrium in the oligomer content or αSyn total protein content in the CSF are associated with PD, amyotrophic lateral sclerosis (ALS), Lewy body dementia (LBD) and Alzheimer's disease (AD). Furthermore, there is new evidence suggesting that these species of aberrant proteins can be transferred from cell to cell and promote the propagation of neurodegeneration in a manner similar to prion. While it has been known for some time that robust, activated microglia-mediated neuroinflammation accompanies the neurodegenerative process in PD, recent studies indicate that extracellular αSyn, particularly the oligomeric forms, plays a key role in inflammatory response, as well as in other neurodegeneration-linked neurotoxic mechanisms.

Amyotrophic lateral sclerosis (ALS) is an incurable neurodegenerative disease that leads to death in a period of between 3 and 5 years after diagnosis. Today, there are no effective treatments for ALS, obtaining preclinical data that support clinical studies for establishing therapies modifying the progression of the disease being a priority for health systems. Today, there is only one approved treatment (Riluzole) for ALS which only achieves rather moderate effects prolonging the survival of the patients between 2-3 months. Of all ALS cases, 90% are sporadic and 10% are familial. Although motor neuron death is responsible for this disease, it has been proven in the last three years that ALS is not a phenomenon exclusively linked to motor neurons since various cell types, among which microglia activation, T-cell infiltration, and acquired immune deficiency response stand out, also influence this disease.

Today, it is known that 20% of familial ALS cases are associated with mutations in the gene encoding the Cu—Zn superoxide dismutase 1 (SOD1) enzyme, one of the main components of the cell defense mechanism against oxidative damage. Furthermore, it is believed that intracellular aggregation and accumulation of SOD1 in motor neurons are crucial for the onset and progression of ALS, although the underlining pathological mechanisms are still unknown.

In recent years, vaccination or immunization has emerged as a potentially powerful therapeutic strategy for the treatment of some neurodegenerative proteinopathies. Despite the fact that some advancements have been made for Alzheimer's disease (AD), this method has been largely unexplored for other diseases such as PD and ALS, today there being only one vaccine under phase I study for PD and none for ALS or other ‘conformational diseases’. In fact, the antibodies produced by immunization with αSyn in a mouse model of PD and passive vaccination with anti-αSyn antibodies in a mouse model of Lewy body disease or α-synuclein transgenic mice have shown encouraging, although modest, results. On the other hand, it has been demonstrated that immunization with highly aggregated α-Syn species in mice with PD aggravates neuroinflammation and neurodegeneration through a dysfunction in regulatory T-cells (Treg) which, as is known, are instrumental in modulating immune response. Generally, although immunization with α-synuclein is currently rated as a very appealing treatment option for PD and other synucleinopathies, current immunotherapy protocols have demonstrated up until now mixed/modestly successful results, and it is clear that new approaches for optimizing vaccination schedules are necessary.

### BRIEF DESCRIPTION OF THE INVENTION

HSPs (chaperones or heat-shock proteins) have two very valuable characteristics that can be productively exploited in a specific manner in order to “intelligently” manipulate the immune response so as to treat ‘conformational’ disorders or ‘proteinopathies’:
the classic chaperone functions thereof; and
the more recently discovered immunoactive properties thereof.

On the basis thereof, the authors of the present invention have combined a peptide, a protein or a polypeptide associated with a ‘conformational disease’ with a biologically active or functional HSP, resulting in a different immunoactive complex which is advantageous for treating the “conformational disease” in question associated with the peptide, protein or polypeptide used.

Therefore, a first aspect of the invention relates to a composition, hereinafter composition of the invention, comprising:

a) a mixture of peptides/polypeptides/proteins comprising i) at least one peptide/polypeptide/protein the amino acid sequence of which corresponds with an aggregating peptide/polypeptide/protein (hereinafter “sequence A of the invention”) or any of the biologically active variants and fragments of said sequence, and ii) a peptide/polypeptide/protein the amino acid sequence of which corresponds with a molecular chaperone (hereinafter “sequence B of the invention”) or with any of the biologically active variants and fragments of said sequence; and/or

b) a fusion peptide/polypeptide/protein, hereinafter “fusion amino acid sequence of the invention”, comprising the amino acid sequences of at least one aggregating peptide/polypeptide/protein (“sequence A of the invention”) or any of the biologically active variants and fragments thereof, and of a peptide/polypeptide/protein the amino acid sequence of which corresponds with a chaperone (“sequence B of the invention”) or with any of the biologically active variants and fragments thereof.

In a preferred embodiment of this aspect of the invention, at least one of the sequences A and/or B are recombinant sequences. In another preferred embodiment, sequence A corresponds with the amino acid sequence of α-synuclein or with any of the biologically active variants and fragments thereof. In another preferred embodiment, sequence B corresponds with the amino acid sequence of the chaperone Grp94 or with any of the biologically active variants and fragments thereof. In another even more preferred embodiment, sequence A corresponds with the amino acid sequence of α-synuclein or with any of the biologically active variants and fragments thereof, and sequence B corresponds with the amino acid sequence of the chaperone Grp94 or with any of the biologically active variants and fragments thereof.

In another preferred embodiment, sequence A corresponds with the amino acid sequence of SOD1 or with any of the biologically active variants and fragments thereof. In another preferred embodiment, peptide B corresponds with the amino acid sequence of the chaperone Hsp70 or with any of the biologically active variants and fragments thereof. In another even more preferred embodiment, sequence A corresponds with the amino acid sequence of SOD1 or with any of the biologically active variants and fragments thereof, and peptide B corresponds with the amino acid sequence of the chaperone Hsp70 or with any of the biologically active variants and fragments thereof

Another aspect of the invention relates to an isolated nucleotide sequence, hereinafter “nucleotide sequence of the invention”, comprising one or more nucleotide sequences encoding a whole fusion protein of the invention or a fraction thereof.

Another aspect of the invention relates to an isolated expression vector (hereinafter “expression vector of the invention”), comprising the nucleotide sequence of the invention.

Another aspect of the invention relates to an isolated host cell (hereinafter “host cell of the invention”), comprising an expression vector of the invention or a nucleotide sequence of the invention.

Another aspect of the invention relates to an antibody or a fragment thereof, hereinafter “antibody of the invention” or “antibody fragment of the invention”, capable of binding to one of sequences A or B of the invention or to the fusion amino acid sequence of the invention. In a preferred embodiment of this aspect, the antibody or the antibody fragment of the invention is obtained after immunization of an animal (in the presence or absence of an adjuvant) or the cells of an animal with a sequence A and B of the invention or with the fusion amino acid sequence of the invention. In a more preferred embodiment, the mammal is human.

Another aspect of the invention relates to an immunotherapeutic cellular composition comprising at least one isolated, activated antigen presenting cell (APC), in which said APC is obtained from a patient diagnosed with a disease caused by an aggregating protein of the invention, and wherein said APC is stimulated by ex vivo exposure to a composition comprising sequence A and sequence B of the invention, or the fusion amino acid sequence of the invention or any of the biologically active variants and fragments thereof.

Another aspect of the invention relates to a pharmaceutical composition, hereinafter “pharmaceutical composition of the invention”, comprising the composition of the invention, the nucleotide sequence of the invention, the expression vector of the invention, the host cell of the invention, the antibody or a fragment of the antibody of the invention and/or the immunotherapeutic cellular composition of the invention. In a preferred embodiment of this aspect, the pharmaceutical composition further comprises a pharmaceutically acceptable vehicle. In another preferred embodiment, the pharmaceutical composition further comprises an adjuvant.

Another aspect of the invention relates to a combined preparation, hereinafter “combined preparation of the invention”, comprising sequence A of the invention and sequence B of the invention.

Another aspect of the invention relates to the use of sequence B of the invention, the composition of the invention, the nucleotide sequence of the invention, the expression vector of the invention, the host cell of the invention, the pharmaceutical composition of the invention, the combined preparation of the invention, or the antibody of the invention, in the preparation of a medicinal product. In a preferred embodiment, the medicinal product is a vaccine.

Another aspect of the invention relates to the use of sequence B of the invention, the composition of the invention, the nucleotide sequence of the invention, the expression vector of the invention, the host cell of the invention, the antibody or a fragment of the antibody of the invention, the immunotherapeutic cellular composition of the invention, the pharmaceutical composition of the invention, or the combined preparation of the invention, in the preparation of a medicinal product for the treatment or prevention of aggregating protein-related diseases or disorders. In a preferred embodiment of this aspect, the aggregating protein-related disease or disorder is selected from the list consisting of: Parkinson's disease (PD), Lewy body dementia (LBD), the Lewy body variant of Alzheimer's disease, multiple system atrophy (MSA), Alzheimer's disease, pure autonomic failure (PAF), amyotrophic lateral sclerosis (ALS), frontotemporal dementia, Gaucher disease, Down syndrome, Huntington's disease, prion disease, Creutzfeldt-Jakob disease and other transmissible spongiform encephalopathies, multiple sclerosis, Gerstmann-Sträussler-Scheinker syndrome, Kuru, fatal familial insomnia, type II diabetes, cerebrovascular amyloidosis, glaucoma, age-related macular degeneration, psychiatric syndromes, schizophrenia and/or schizophrenia-like disorders, and neurodegeneration due to age-related protein aggregation. More preferably, it is Parkinson's disease (PD) and amyotrophic lateral sclerosis (ALS).

Another aspect of the invention relates to the use of sequences A and B of the invention, the composition of the invention, the nucleotide sequence of the invention, the expression vector of the invention, the host cell of the invention, the pharmaceutical composition of the invention, the combined preparation of the invention, or the antibody of the invention, in the preparation of a medicinal product. In a preferred embodiment, the medicinal product is a vaccine.

Another aspect of the invention relates to the use of sequences A and B of the invention, the composition of the invention, the nucleotide sequence of the invention, the expression vector of the invention, the host cell of the invention, the antibody or a fragment of the antibody of the invention, the immunotherapeutic cellular composition of the invention, the pharmaceutical composition of the invention, or the combined preparation of the invention, in the preparation of a medicinal product for the treatment or prevention of aggregating protein-related diseases or disorders. Preferably, the aggregating protein is α-synuclein. In a preferred embodiment of this aspect, the α-synuclein-related disease or disorder or the disease or disorder related with another aggregating protein is selected from the list consisting of: Parkinson's disease (PD), Lewy body dementia (LBD), the Lewy body variant of Alzheimer's disease, multiple system atrophy (MSA), Alzheimer's disease, pure autonomic failure (PAF), amyotrophic lateral sclerosis (ALS), frontotemporal dementia, Gaucher disease, Down syndrome, Huntington's disease, prion disease, Creutzfeldt-Jakob disease and other transmissible spongiform encephalopathies, multiple sclerosis, Gerstmann-Sträussler-Scheinker syndrome, Kuru, fatal familial insomnia, type II diabetes, cerebrovascular amyloidosis, glaucoma, age-related macular degeneration, psychiatric syndromes, schizophrenia and/or schizophrenia-like disorders, and neurodegeneration due to age-related protein aggregation. More preferably, it is Parkinson's disease (PD) and amyotrophic lateral sclerosis (ALS).

In another preferred embodiment, the α-synuclein-related disease or disorder is selected from the list consisting of: Parkinson's disease (PD), Lewy body dementia, multiple system atrophy (MSA), Gaucher disease, pure autonomic failure (PAF) and Alzheimer's disease.

Another even more preferred embodiment relates to the use of a composition of the invention comprising as sequence A, the amino acid sequence of α-synuclein, and as sequence B, the amino acid sequence of the chaperone Grp94, for the preparation of a medicinal product for the treatment or prevention of an α-synuclein-related disease or disorder. In a preferred embodiment, the α-synuclein-related disease or disorder is selected from the list consisting of: Parkinson's disease (PD), Lewy body dementia, multiple system atrophy (MSA), Gaucher disease, pure autonomic failure (PAF) and Alzheimer's disease.

Another even more preferred embodiment relates to the use of a composition of the invention comprising as sequence A, the amino acid sequence of α-synuclein, and as sequence B, the amino acid sequence of the chaperone Grp94.

Another aspect of the invention relates to the use of the combined preparation of the invention, comprising sequence A and sequence B of the invention, in the preparation of a medicinal product for combined, simultaneous or sequential administration, for the treatment or prevention of aggregating protein-related diseases or disorders. More preferably, the aggregating protein is α-synuclein or α-synucleinopathies. In a preferred embodiment of this aspect, the α-synuclein-related disease or disorder is selected from the list consisting of: Parkinson's disease (PD), Lewy body dementia (LBD), the Lewy body variant of Alzheimer's disease, multiple system atrophy (MSA), Alzheimer's disease, amyotrophic lateral sclerosis (ALS), Down syndrome, frontotemporal dementia, Gaucher disease, Huntington's disease, prion disease, Creutzfeldt-Jakob disease and other transmissible spongiform encephalopathies, multiple sclerosis, Gerstmann-Sträussler-Scheinker syndrome, Kuru, fatal familial insomnia, type II diabetes, cerebrovascular amyloidosis, glaucoma, age-related macular degeneration, psychiatric syndromes, schizophrenia and/or schizophrenia-like disorders, and neurodegeneration due to age-related protein aggregation. More preferably, it is Parkinson's disease.

Another even more preferred embodiment relates to the use of the combined preparation of the invention comprising as sequence A, the amino acid sequence of α-synuclein, and as sequence B, the amino acid sequence of the chaperone Grp94, for the preparation of a medicinal product for the treatment or prevention of an α-synuclein-related disease or disorder. In a preferred embodiment, the α-synuclein-related disease or disorder is selected from the list consisting of: Parkinson's disease (PD), Lewy body dementia (LBD), the Lewy body variant of Alzheimer's disease, multiple system atrophy (MSA), Alzheimer's disease, amyotrophic lateral sclerosis (ALS), frontotemporal dementia, Gaucher disease, Down syndrome, Huntington's disease, prion disease, Creutzfeldt-Jakob disease and other transmissible spongiform encephalopathies, multiple sclerosis, Gerstmann-Sträussler-Scheinker syndrome, Kuru, fatal familial insomnia, type II diabetes, cerebrovascular amyloidosis, glaucoma, age-related macular degeneration, psychiatric syndromes, schizophrenia and/or schizophrenia-like disorders, and neurodegeneration due to age-related protein aggregation. More preferably, it is Parkinson's disease (PD) and amyotrophic lateral sclerosis (ALS).

### DESCRIPTION OF THE DRAWINGS

FIG. 1 shows the evaluation of the effect on murine splenocytes stimulated in vitro with an αSyn and Grp94 combination. A. Activation profile flow cytometry analysis of murine splenocytes in cultures originating from healthy C57BL/6 mice, incubated in vitro for 24 hrs with LPS (0.5 μg/ml), αSyn (14.3 nM or 0.2 μg/ml), Grp94 (14.3 nM or 1.3 μg/ml), an αSyn and Grp94 combination (0.2 μg/ml and 1.3 μg/ml, respectively), or with buffer solution as reference. B-D. Levels of IFN-γ (B), IL-10 (C) and IL-17 (D) in the supernatants of the cultures mentioned in (A), measured using ELISA. The graphs are representative of two independent experiments, and the values correspond to the average ±S.D. of duplicates.

FIG. 2 shows the Immune response generated in C57BL/6 mice immunized with an αSyn and Grp94 combination according to protocol 1. A. Flow cytometry analysis of the CD4⁺/CD25^{h}/Foxp3⁺ (Treg) splenocyte population with respect to the total CD4⁺ splenocytes. B and C. Levels of IFN-γ (B) and IL-10 (C) in the supernatants of splenocyte cultures incubated in vitro for 24 h with αSyn (15 μg/ml) or buffer solution. D. Relative levels of anti-αSyn IgG antibodies with respect to the total IgG in serum, measured using ELISA. The values correspond to the average ±S.D. with samples from 5 mice per group (N=5).

FIG. 3 shows the immune response and behavioral test in C57BL/6 splenocyte receptor mice (obtained after the immunization of mutant mice according to protocol 1) with subsequent Parkinson's disease modeling by means of administration of MPTP. A and B. Flow cytometry analysis of the CD4⁺/CD25^{h} (Treg) splenocyte population with respect to the total CD4⁺ splenocytes (A), and of the CD4⁺/CD25⁻ (Teff) population with respect to the total CD4⁺ splenocytes (B). C and D. Levels of IFN-g (B) and IL-10 (C) in the supernatants of splenocyte cultures incubated in vitro for 24 hrs with αSyn (15 μg/ml) or LPS (0.5 μg /ml). E. Footprinting behavioral test (on front paws) after the adoptive transfer and subsequent administration of MPTP for 3 months, a recovery period of 1 month and administration of amphetamines (see Materials and Methods). The ‘Saline’ and ‘MPTP’ groups correspond to the ‘healthy’ and ‘sick’ animal controls which have not adopted splenocytes. The values correspond to the average±S.E.M. of each group (N=7, with the exception of ‘Saline’ with N=4, and ‘MPTP’ with N=5).

FIG. 4 shows the immune response generated in C57BL/6 mice immunized with an αSyn and Grp94 combination, according to protocol 2. A and B. Flow cytometry analysis of the CD4⁺/CD25h (Treg) splenocyte population with respect to the total CD4⁺ splenocytes (A), and of the CD4⁺/CD25⁻ (Teff) population with respect to the total CD4⁺ splenocytes (B). C. Levels of IFN-γ in the supernatants of splenocyte cultures incubated in vitro for 24 hrs with αSyn (15 μg/ml) or buffer solution. D. Relative levels of anti-αSyn IgG antibodies with respect to the total IgG in serum, measured using ELISA. E. Levels of anti-αSyn IgM antibodies in serum, measured using ELISA. The values correspond to the average ±S.E.M. with samples from 5 mice per group (N=5).

FIG. 5 shows the behavioral test in C57BL/6 splenocyte receptor mice (obtained after immunization of the donor mice according to protocol 2) with subsequent Parkinson's disease modeling by means of administration of MPTP. Footprinting behavioral test (on rear paws) after the adoptive transfer of splenocytes, and subsequent administration of MPTP for 3 months, a recovery period of 1 month and administration of amphetamines (see Materials and Methods). The ‘Saline’ and ‘MPTP’ groups correspond to the ‘healthy’ and ‘sick’ animal controls which have not adopted splenocytes. The values correspond to the average ±S.E.M. of each group (N=5).

FIG. 6 shows the immune response generated in mice model of ALS (Tg SOD1) vaccinated with a SOD1 and Hsp70 combination according to protocol 4. Preliminary results. Flow cytometry analysis of the CD4⁺ splenocyte population (A), CD4⁺/CD25^{h}/Foxp3⁺ (Treg) (B), and CD4⁺CD25⁻ (Teff) (C), with respect to the total CD4⁺ splenocytes. D. Levels of IFN-γ in the supernatants of splenocyte cultures incubated in vitro for 24 hrs with SOD1 (20 μg/ml) or buffer solution. E and F. Paired ratio per mouse of total IgG1/IgG2a antibody level (E) or anti-SOD1 level (F) in serum, measured using ELISA. Non-vehicle: non-transgenic animal (healthy control). The values correspond to the average ±S.E.M. with samples from 3 mice per group (N=3).

FIG. 7 shows the evaluation of the degree of protection in the murine model of ALS (Tg SOD1) by means of measuring the weight and survival of the ‘animals vaccinated according to protocol 4. Preliminary results. A. Representation of the progression of the body weight over time with respect to the maximum weight reached for each mouse. B. Representation of the survival with respect to time (Kaplan-Meier graph). Non-vehicle: non-transgenic animal (healthy control). Number of animals per group: 3 (N=3).

FIG. 8 shows the characterization of the immune response generated by immunization of C57BL/6 mice with a αSyn and Hsp70 combination/complex in the absence of adjuvant. A. Flow cytometry analysis of the CD4⁺/CD25⁺/Foxp3⁺ (Treg) splenocyte population. B. Anti-αSyn IgG antibody level measured in serum using ELISA. C. Representation of the paired ratio per mouse of the levels of IL-10/IFN-γ measured in serum, using ELISA. αS: α-synuclein (αSyn); ‘70’: Hsp70; ‘low’: low dose; ‘high’: high dose (see Materials and Methods). The values correspond to the average ±S.E.M. with samples from 6-7 mice per group (N=6-7).

### DETAILED DESCRIPTION OF THE INVENTION

The authors of the present invention have found that immunization with mixtures of α-synuclein (αSyn) and molecular chaperone are capable of promoting, modulating and redirecting immune response, being able to be very beneficial for the development of new therapeutic and prophylactic strategies against synucleinopathies. Additionally, the authors of the present invention have also proven how the use of other aggregating proteins in combination with molecular chaperones are shown to be excellent therapeutic tools for immunization against various “proteinopathies”, such as Alzheimer's disease (AD), Huntington's disease, prion diseases, and type II diabetes.

There are provided, therefore, antibodies and cells (passive immunization) and vaccines (active immunization) for use in several methods for diagnosing and combating (which includes delaying the onset, the treatment and/or the prevention of) aggregating protein-related disorders, and more preferably α-synuclein-related disorders or α-synucleinopathies, such as Parkinson's disease (PD), Lewy body dementia (LBD), the Lewy body variant of Alzheimer's disease, multiple system atrophy (MSA) and other α-synuclein-related neurodegenerative disorders. Furthermore, the antibodies, cells and vaccines can be used for delaying the onset, the treatment and/or prevention of other neurodegenerative disorders such as Alzheimer's disease, Down syndrome, amyotrophic lateral sclerosis (ALS), frontotemporal dementia, Gaucher disease, Huntington's disease, prion disease, Creutzfeldt-Jakob disease, multiple sclerosis, Gerstmann-Sträussler-Scheinker syndrome, Kuru, fatal familial insomnia, cerebrovascular amyloidosis, glaucoma, age-related macular degeneration, psychiatric syndromes, schizophrenia and/or schizophrenia-like disorders, neurodegeneration due to age-related protein aggregation. The antibodies, cells and vaccines can also be used in detection methods for the purpose of diagnostic therapy or follow up of said diseases, among others.

### Compositions of the Invention

Therefore, a first aspect of the invention relates to a composition, hereinafter composition of the invention, comprising:

a) a mixture of peptides/polypeptides/proteins comprising i) at least one peptide/polypeptide/protein the amino acid sequence of which corresponds with an aggregating peptide/polypeptide/protein (hereinafter “sequence A of the invention”) or any of the biologically active variants and fragments of said sequence, and ii) a peptide/polypeptide/protein the amino acid sequence of which corresponds with a molecular chaperone (hereinafter “sequence B of the invention”) or with any of the biologically active variants and fragments of said sequence; and/or

b) a fusion peptide/polypeptide/protein, hereinafter “fusion amino acid sequence of the invention”, comprising the amino acid sequences of at least one aggregating peptide/polypeptide/protein (“sequence A of the invention) or any of the biologically active variants and fragments thereof, and of a peptide/polypeptide/protein the amino acid sequence of which corresponds with a chaperone (“sequence B of the invention”) or with any of the biologically active variants and fragments thereof.

“Sequence A” or “aggregating peptide” and/or “aggregating protein” is understood herein as peptides, polypeptides or proteins commonly found forming aggregates in ‘conformational’ diseases, particularly in neurodegenerative diseases (Knowles T P et al. 2014. Nat Rev Mol Cell Biol. 15:384-96.). These proteins include, but are not limited to, α-synuclein (SEQ ID NO: 1), huntingtin (SEQ ID NO: 2), beta-amyloid peptide (SEQ ID NO: 3), TDP-43 (SEQ ID NO: 4), FUS (SEQ ID NO: 5), tau (SEQ ID NO: 6), prion protein (SEQ ID NO: 7), fibronectin (SEQ ID NO: 8), and superoxide dismutase or SOD1, (SEQ ID NO: 9).

Therefore, in a preferred embodiment of this aspect of the invention the aggregating protein or sequence A of the composition of the invention is selected from the list consisting of α-synuclein, huntingtin, beta-amyloid peptide, TDP-43, FUS, tau, prion protein, fibronectin, SOD1 or any of the combinations thereof.

More preferably, the aggregating protein is α-synuclein or any of the biologically active variants and fragments thereof.

In another preferred embodiment, the aggregating protein is SOD1 or any of the biologically active variants and fragments thereof.

α-synuclein (SNCA, NACP, PARK1, PARK4, PD1, alphα-synuclein; non A-beta component of AD amyloid; synuclein alpha-140) is understood herein as a member of the synuclein family which also includes beta- and gamma-synuclein. Synucleins are expressed abundantly in the brain and alpha- and beta-synuclein D2 selectively inhibit phospholipase. α-synuclein can be used for integrating the signaling and the traffic of the presynaptic membrane. α-synuclein defects have been involved in the pathogenesis of Parkinson's disease. α-synuclein is a main component of amyloid plaques in the brain of patients with Alzheimer's disease. Two different isoforms have been identified for this gene. Its amino acid sequence (the amino acid sequence of α-synuclein) corresponds with SEQ ID NO: 1.

In the context of the present invention, the α-synuclein gene is also defined by a nucleotide or polynucleotide sequence, which constitutes the coding sequence of the protein included in SEQ ID NO: 1 and would comprise different variants originating from:

a) nucleic acid molecules encoding a polypeptide comprising the amino acid sequence of SEQ ID NO: 1,

b) nucleic acid molecules the complementary strand of which hybridizes with the polynucleotide sequence of a),

c) nucleic acid molecules the sequence of which differs from a) and/or b) due to genetic code degeneration,

d) nucleic acid molecules encoding a polypeptide comprising the amino acid sequence with an identity of at least 80%, 90%, 95%, 98% or 99% with SEQ ID NO: 1, and in which the polypeptide encoded by said nucleic acids has the activity and structural characteristics of the α-synuclein protein. Immunogenic activity is also included within this activity.

In addition to using the recombinant protein or synthetic peptides, α-synuclein can be derived directly from Lewy bodies present in the post mortem autopsy of human brain tissue for cases with α-synucleinopathies. Both soluble (i.e., preparations soluble in Tris-buffered saline solution) and insoluble (i.e., preparations insoluble in Tris-buffered saline solution) α-synuclein fractions are purified. These sample preparations can be injected into animals as such, or fractioned using chromatographic methods before injection. The generated antibodies can be used for the treatment of patients in a passive vaccination schedule or in diagnostic immunoassays.

In the present invention, α-synuclein refers both to the soluble, unfolded native form and to that with conformational and post-translational modifications. Furthermore, native α-synuclein can be modified by means of, for example, but without limitation, substitution of Ser/Ala with Cys, for example, through site-directed mutagenesis. An example of this type is described in the following patent application by Chang, US2006/0018918 A1.

SOD1 (also referred to as ALS; SOD; ALS1; IPOA; hSod1; HEL-S-44; homodimer) is understood herein as a protein that binds copper and zinc ions and is one of two isozymes responsible for destroying free superoxide radicals in the body. The encoded isozyme is a soluble cytoplasmic protein acting as a homodimer to convert natural but damaging superoxide radicals into molecular oxygen and hydrogen peroxide. The other isozyme is a mitochondrial protein. Mutations in this gene were involved as causes of familial amyotrophic lateral sclerosis. Its amino acid sequence (the amino acid sequence of SOD1) is included in GenBank sequence NP_000445.1, NM_000454.4.

In the context of the present invention, the SOD1 gene is also defined by a nucleotide or polynucleotide sequence which constitutes the coding sequence of the protein listed in sequence NP_000445.1 and would comprise different variants originating from:

a) nucleic acid molecules encoding a polypeptide comprising the amino acid sequence NP_000445.1,

b) nucleic acid molecules the complementary strand of which hybridizes with the polynucleotide sequence of a),

c) nucleic acid molecules the sequence of which differs from a) and/or b) due to genetic code degeneration,

d) nucleic acid molecules encoding a polypeptide comprising the amino acid sequence with an identity of at least 80%, 90%, 95%, 98% or 99% with NP_000445.1, and in which the polypeptide encoded by said nucleic acids has the activity and structural characteristics of the SOD1 protein. Immunogenic activity is also included within this activity.

In another preferred embodiment of this aspect of the invention, the chaperone or sequence B of the invention belongs to the Hsp70 family, Hsp90 family, immunophilin family, peptidase C56 family, PPlase family, 14-3-3 family, the small heat-shock protein group, small GTPase family, Hsp40 (DnaJ) family, or any of the combinations thereof. More preferably, the chaperones of the Hsp90 family are selected from Grp94 (SEQ ID NO: 10) and Hsp90 (SEQ ID NO: 11) or any of the combinations thereof. In another preferred embodiment, the chaperones of the Hsp70 family are selected from Hsp70 (SEQ ID NO: 12), Hsc70 (SEQ ID NO: 13) and Grp75 (SEQ ID NO: 14) or any of the combinations thereof. In another preferred embodiment, the chaperones of the immunophilin family are selected from FKBP12 (SEQ ID NO: 15) and FKBP4/FKBP52 (SEQ ID NO: 16) or the combination thereof. In another preferred embodiment, the chaperone of the peptidase C56 family is DJ1/PARK7 (SEQ ID NO: 17). In another preferred embodiment, the chaperones of the PPlase family are selected from Pin1 (SEQ ID NO: 18), CypA (SEQ ID NO: 19), Cyp40 (SEQ ID NO: 20) or any of the combinations thereof. In another preferred embodiment, the chaperones of the 14-3-3 family are selected from 14-3-3 gamma (SEQ ID NO: 21), 14-3-3 epsilon (SEQ ID NO: 22), 14-3-3 tau (SEQ ID NO: 23) or any of the combinations thereof. In another preferred embodiment, the chaperone of the small heat-shock protein group is Hsp27 (SEQ ID NO: 24). In another preferred embodiment, the chaperone of the small GTPase family is Rab11A (SEQ ID NO: 25). In another preferred embodiment, the chaperones of the Hsp40 (DnaJ) family are selected from Hsp40 (SEQ ID NO: 26) and CSP (SEQ ID NO: 27) or the combination thereof. In another preferred embodiment, the chaperone is clusterin (SEQ ID NO: 28), Grp170 (SEQ ID NO: 29), calreticulin (SEQ ID NO: 30), Hsp105 (SEQ ID NO: 31), CHIP (SEQ ID NO: 32), alpha-crystallin (SEQ ID NO: 33) or any of the combinations thereof. In the composition of the invention, the combination of different chaperones, even from different group or family, can also be possible.

In a more preferred embodiment, the chaperone is Grp94 or any of its biologically active variants, and combinations thereof. Even more preferably, the chaperone is Grp94.

Grp94 (heat shock protein 90 kDa beta (Grp94), member 1, ECGP; GP96; TRA1; GRP94) is a gene encoding a member of family of adenosine triphosphate (ATP)-metabolizing molecular chaperones with roles in stabilizing and folding other proteins. The encoded protein is localized to melanosomes and the endoplasmic reticulum. Expression of this protein is associated with a variety of pathogenic states, including tumor formation. There is a microRNA gene located within the 5′ exon of this gene. There are pseudogenes for this gene on chromosomes 1 and 15.

In the context of the present invention, the Grp94 gene is also defined by a nucleotide or polynucleotide sequence, which constitutes the coding sequence of the protein included in SEQ ID NO: 10 and would comprise different variants originating from:

a) nucleic acid molecules encoding a polypeptide comprising the amino acid sequence of SEQ ID NO: 10,

b) nucleic acid molecules the complementary strand of which hybridizes with the polynucleotide sequence of a),

c) nucleic acid molecules the sequence of which differs from a) and/or b) due to genetic code degeneration,

d) nucleic acid molecules encoding a polypeptide comprising the amino acid sequence with an identity of at least 80%, 90%, 95%, 98% or 99% with SEQ ID NO: 10, and in which the polypeptide encoded by said nucleic acids has the activity and the structural characteristics of the Grp94 protein.

Several amino acid sequences are available in the databases for the peptides and proteins of the invention, differing in one or more amino acids of the following amino acid sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33. These peptide and protein variants are comprised in the present invention, provided that they have an activity and a structural behavior similar to the amino acid sequence from which it is derived. As it is used herein, the protein variants of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33 are proteins with an amino acid sequence differing from said amino acid sequence due to one or more substitutions, additions or deletions of amino acids. Generally, any amino acid residue of the sequence shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33 can be substituted with another amino acid, provided that the resulting protein variant exhibits an activity and a structural behavior similar to the amino acid sequence from which it is derived. The activity and structural behavior of said proteins can be determined using methods known in the state of the art. In particular, protein variants differing from the amino acid sequences that have been shown in up to 5, 10, 15, 20 or even 25 amino acid positions are included.

Preferably, the substitutions are conservative substitutions, i.e., substitutions of an amino acid residue with another amino acid having a similar polarity, acting as a functional equivalent. Preferably, the amino acid residue used as a substitute is selected from the same amino acid group as the amino acid residue to be substituted. For example, a hydrophobic residue can be substituted with another hydrophobic residue, or a polar residue can be substituted with another polar residue having the same charge. Functionally homologous amino acids which can be used for a conservative substitution comprise, for example, non-polar amino acids such as glycine, valine, alanine, isoleucine, leucine, methionine, proline, phenylalanine and tryptophan. The examples of uncharged polar amino acids include serine, threonine, glutamine, asparagine, tyrosine and cysteine. The examples of charged polar amino acids (basic) include histidine, arginine and lysine. The examples of charged polar amino acids (acidic) include aspartic acid and glutamic acid.

The term “variant” also comprises amino acid sequences including more amino acids than the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33, i.e., amino acid sequences in which one or more amino acids have been inserted. Such insertions can in principle take place in any position of the amino acid sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33. The insertions can be contiguous amino acid segments comprising, for example, 2, 3, 4, 5, 6, 7, 8, 9 or 10 amino acids. Similarly, the term “variant” also includes amino acid sequences in which one or more amino acids are deleted compared with the amino acid sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33. Deletion can involve several contiguous amino acid residues of the mentioned sequences for example, 2, 3, 4, 5, 6, 7, 8, 9 or 10 amino acids. This deletion does not affect the activity and structural behavior of said variants. The protein variants can also include structural modifications, for example, modified amino acids, such as amino acids which have been altered by phosphorylation, nitration, glycosylation, acetylation, thiolation, branching and/or cyclization.

The protein variants of sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33, which have been modified by substitution, addition or deletion of amino acids, usually show a considerable degree of amino acid sequence identity or of identity with the protein of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33. Preferably, the homology or identity at the amino acid level is at least 70%, 75%, 80%, 85%, 90%, 91%, 92%, 93%, 94%, 95%, 96%, 97%, 98% or 99% if the sequence of the variant is optimally aligned with that of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33, respectively. The methods and computer programs for determining amino acid homology are well known in the art.

The context of the invention also contemplates using immunologically active fragments either derived from the protein of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33, or from variants thereof. Immunologically active fragments of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33 or variants thereof include peptides or polypeptides differing from the amino acid sequence shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33, or of variants thereof due to the absence of one or more amino acids at the N-terminal end and/or the C-terminal end of the protein. The person having average skill will be capable of determining other active fragments of the sequence represented in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and/or SEQ ID NO: 33 or variants thereof by means of performing the routine methods described in great detail in the state of the art.

Within biologically active variants, it is understood, however, that a protein activity loss of 20%, 30%, 40%, 50%, 60%, 70%, or even up to 80% can be tolerated if a sequence (A and/or B) or a particular fusion amino acid sequence exhibits other favorable characteristics, such as greater stability or shelf life or improved pharmacological properties. A person skilled in the art will be capable of easily determining if a given fusion amino acid sequence of the invention is suitable for being used in a desired therapeutic context.

In a more preferred embodiment of the invention, the aggregating protein and/or the chaperone were obtained in a recombinant manner. Alternatively, both the aggregating protein and the chaperone can be obtained from cells or tissues. In turn, both the aggregating protein/proteins and the chaperone or chaperones forming part of the composition of the invention can be of a human or animal origin, being able to be any known ortholog of said protein or proteins. Preferably, it is of human origin (or in the case of having been obtained in a recombinant manner, the amino acid sequence has a high percentage of identity with the known amino acid sequence of the human protein, preferably of at least 70%, more preferably 80%, 90%, 95%, 98%, or much more preferably 90%, with the known human amino acid sequence of said protein).

As it is used herein, the term “identity” refers to the proportion of identical amino acids (or nucleotides) between two amino acid (or nucleotide) sequences that are compared.

When comparing the sequence of an aggregating protein or a human chaperone with the sequence of the same protein in other species, it is evident that there are certain regions that are more conserved than others. This information can be indicative of the fact that those zones are crucial for maintaining protein structure or function. Both the active regions determining the activity of said proteins and the conserved cysteines between which disulfide bridges are established, important for protein formation, will most probably be conserved between both proteins, whereas divergence is more apparent in other regions.

As it is used herein, the term “homology” refers to the similarity between two structures due to a common evolutionary lineage, and more specifically, to the similarity between the amino acids (or nucleotides) of two or more proteins (polynucleotides) or amino acid (or nucleotide) sequences.

Given that two proteins are considered homologous if they have the same evolutionary origin or if they have similar function and structure, it is generally assumed that values of similarity or identity greater than 30% indicate homologous structures. Therefore, it can be considered that percentages of identity of at least 80% will include the active regions conserved between the different species capable of generating immunogenicity or having chaperone activity and also immunological activity, where appropriate.

Sequence A and sequence B of the invention can be found bound in a fusion protein, or alternatively chemically bound to one another, for example, but without limitation, by means of cross-linking.

“Cross-linking” is understood herein as a chemical technique binding two or more molecules by means of a covalent bond; they contain substances having ends reactive with respect to specific functional groups of proteins or other molecules. These can be divided into homobifunctional (the same reactive groups) and heterobifunctional (different reactive groups), having chemical cross-linkers which may or may not be inverted. Homobifunctional cross-linkers have a potential drawback connecting two neighboring groups, either on the surface of nanoparticles or in the induction of undesired cross-linking proteins. Heterobifunctional cross-linkers allow sequential conjugations, minimizing polymerization. For example, sulfosuccinimidyl 4-(N-maleimidomethyl)cyclohexane-1-carboxylate (sulfo-SMCC) can be used for coupling the content of thiol biomolecules with amine-coated nanoparticles, or vice versa, considering that the heterobifunctional cross-linker 1-ethyl-3-(3-dimethylaminopropyl)carbodiimide (EDC) is commonly used for connecting NH₂ and —COOH groups (Jameson and Wong, 2009; Hurt et al., 2010 Chem Biol. April 24; 16(4): 372-381).

Additionally, sequences A and B of the invention can be obtained synthetically. The design of synthetic amino acid sequences is known in the state of the art. Therefore, analysis of the data of experimentally determined antigenic sites has revealed that hydrophobic residues, Leu and Val, if they are found on the protein surface, have a great tendency to form part of antigenic determinants. Semi-empirical methods using the physicochemical properties of the amino acid residues and their frequencies and occurrence in segments of experimentally known epitopes for determining the antigenic determinants of a protein have therefore been developed.

Synthetic amino acid sequences have several advantages with respect to specific antibody production and reactivity. The exact sequence of the synthesized peptide can be selected from the amino acid sequence of the protein as determined by the amino acid sequence of the protein or by the predicted amino acid sequence determined from the DNA sequence encoding the protein.

The use of synthetic specific peptides eliminates the need for a full-length protein in vaccination and production of an antibody assay. Furthermore, Merrifield solid-phase peptide synthesis techniques allow chemically producing unlimited amounts of peptides, essentially from the synthetic peptide of interest. Said techniques have progressed to the automated peptide synthesis capacity.

### Nucleotides, Gene Constructs, Expression Vectors and Host Cells of the Invention

Synthetic amino acid sequences can also be prepared through expression in a host cell containing a recombinant DNA molecule comprising a nucleotide sequence that is transcribed to the peptides or proteins of the invention, operatively bound to an expression control sequence, or a vehicle or recombinant DNA cloning vector containing such recombinant DNA molecule. Alternatively, the peptides can be expressed by direct injection of a single DNA molecule into a host cell. Generally, the synthetic peptides produced according to the invention have protective antigenic sequences. As it is used herein, the expression “protective antigen” defines those antigens capable of generating a protective immune (immunogenic) response in the host, i.e., a host response, leading to the generation of immune effector molecules, antibodies or cells sterilizing, suppressing or reducing the activity of the aggregating proteins which are causing a disease, thereby “protecting” the host from a clinical or sub-clinical disease and/or from loss of productivity. Such protective immune response can usually take place through the generation of an immunocompetent cell-mediated protective response and of antibodies which are capable of inhibiting the function, cytotoxicity mechanism or cell-to-cell propagation of aggregating proteins, leading to their elimination or to a loss of their negative effect on the host. Said host is preferably a mammal, and even much more preferably, a human.

Therefore another aspect of the invention relates to a nucleotide sequence, hereinafter nucleotide sequence of the invention, comprising one or more nucleotide sequences, encoding the entire or a fraction of sequence A, sequence B, or the fusion amino acid sequence of the invention.

Providing a nucleic acid molecule or a nucleotide sequence according to the invention therefore makes it possible to obtain sequences A and B, and/or the fusion amino acid sequence/sequences of the invention, as well as their biologically active variants or immunogenic fragments, in amounts not available up until now, thereby allowing the development of compositions, preferably pharmaceutical compositions.

This aspect of the invention provides a method for preparing a recombinant protein or peptide encoded by a nucleic acid molecule or nucleotide sequence of the invention which comprises culturing a eukaryotic or prokaryotic cell containing a nucleic acid molecule or nucleotide sequence of the invention, in conditions in which said protein or peptide is expressed, and recovering said peptide thus produced.

This method includes the cloning and expression vectors comprising the nucleic acid molecules or nucleotide sequences of the invention. Such expression vectors include suitable control sequences, such as, for example, translation control elements (such as start and stop codes) and transcription control elements (for example, promoter-operator regions, binding sites, etc.). The vectors according to the invention can include plasmids and viruses (comprising bacteriophages and eukaryotic viruses), according to methods well known and documented in the art, and can be expressed in a variety of different expression systems, also well known and documented in the art. Suitable viral vectors include baculoviruses and also adenoviruses and vaccine viruses. Many other viral and non-viral vectors are described and known in the art.

Therefore, another aspect of the invention relates to an isolated expression vector (hereinafter expression vector of the invention), comprising the nucleotide sequence of the invention.

A variety of techniques which can be used for introducing such vectors into prokaryotic or eukaryotic cells for expression, or into a germline or into somatic cells, to form transgenic animals, is also known. Suitable transformation or transfection techniques are well described in the bibliography.

Transformed or transfected eukaryotic or prokaryotic host cells containing a nucleic acid molecule or the nucleotide sequence according to the invention, as defined above, also form part of this aspect of the invention.

Therefore, another aspect of the invention relates to an isolated host cell, (hereinafter host cell of the invention), comprising an expression vector of the invention or a nucleotide sequence of the invention.

### Antibodies of the Invention

The compositions of the invention are formulated for use as immunogens. These immunogens can also be used as vaccines in animals, and more particularly in mammals, including humans, or for producing a response in the production of antibodies in animals. For the formulation of such compositions, an immunologically effective amount of each of the sequences A and B of the composition of the invention together with the use of a suitable physiologically acceptable vehicle for administration to mammals including humans, are necessary. Sequences A and B can be covalently bound to one another, to other peptides, to a vehicle protein or to other vehicles, incorporated in nanoparticles, liposomes or other similar vesicles, and/or mixed with an adjuvant or absorbent as is well known in the field of vaccines. For example, sequences A and B can be mixed with immunostimulating complexes. Alternatively, sequences A and B are not coupled to and are merely mixed with a physiologically acceptable vehicle such as a normal saline or buffer compound suitable for administration to mammals including humans.

Therefore, and as described above, synthetic sequences A and B produced according to the invention have protective antigenic sequences. These protective antigens are capable of generating a protective immune (immunogenic) response in the host, i.e., a host response, leading to the generation of immune effector molecules, antibodies or cells inactivating or reducing the activity or effectiveness of the aggregating proteins causing a disease, thereby “protecting” the host from a clinical or sub-clinical disease and from loss of productivity. Such protective immune response can usually take place through the generation of immunocompetent cell populations and/or antibodies which are capable of inhibiting the function of the aggregating protein causing the disease.

As with all immunogenic compositions for producing a response in antibodies, the immunologically effective amounts of sequences A and B of the invention must be determined empirically. Considered factors include the immunogenicity of the natural peptide, whether or not the peptide is complexed through a covalent bond with an adjuvant or a vehicle protein or another vehicle, and the route of administration for the composition, for example, and without limitation, the intravenous route, intramuscular route, subcutaneous route, and as in a particular embodiment of the invention, the intranasal route or oral route, as well as the number of immunization doses that would be administered. Such factors are known in the field of vaccines and it is in accordance with the skill of the immunologist who has performed such determinations without undue experimentation. The antibodies will be capable of binding to sequences A or B of the invention or to the fusion amino acid sequence of the invention.

Therefore, another aspect of the invention relates to an antibody or a fragment thereof, hereinafter antibody of the invention or antibody fragment of the invention, capable of binding to one of sequences A or B of the invention or to the fusion amino acid sequence of the invention. In a preferred embodiment of this aspect, the antibody or the antibody fragment of the invention is obtained after immunization of an animal or of the cells of an animal with amino acid sequences A and B of the invention or with the fusion amino acid sequence of the invention. In a particular embodiment of this aspect of the invention, the animal which is used for immunization is a mammal, including human beings.

In a preferred embodiment, the animal which is used for immunization is a mammal, not including human beings.

These antibodies can be monoclonal or polyclonal. The antibody of the invention can be also recombinant, chimeric, humanized, synthetic or a combination of any of the foregoing.

In another particular embodiment of this aspect of the invention, the antibodies are used as a medicinal product.

The antibodies of the present invention can be formulated for administration to an animal, and more preferably to a mammal, including human beings, in a variety of forms. Therefore, the antibodies can be in a sterile aqueous solution or in biological fluids, such as serum. The aqueous solutions may or may not be buffered and have additional active or inactive components. The additional components include salts for modulating ionic strength, preservatives including, but not limited to, antimicrobial agents, antioxidants, chelating agents and the like, and nutrients including glucose, dextrose, nucleotides, vitamins and minerals. Alternatively, the antibodies can be prepared for administration in solid form. The antibodies can be combined with several inert excipients or vehicles, including but not limited to, binders such as microcrystalline cellulose, tragacanth gum or gelatin; excipients such as starch or lactose; dispersing agents such as alginic acid or cornstarch; lubricants such as magnesium stearate, glidants such as colloidal silicon dioxide; sweetening agents such as sucrose or saccharine; or favoring agents such as mint or methyl salicylate.

The antibodies or their formulations can be administered to an animal, including a mammal and, therefore, human beings, in a many ways. Such ways include, but are not limited to, intraperitoneally, intravenously, intramuscularly, subcutaneously, intrathecally, intraventricularly, orally, enterally, parenterally, intranasally or cutaneously.

The antibody dosage for obtaining a pharmaceutically effective amount depends on a variety of factors, such as, for example, the age, weight, gender, tolerance, etc. . . . of the animal.

The term “antigen” refers herein to a cell surface molecule (generally a protein or a polysaccharide) which can induce antibody formation. There are many different types of molecules which can act as antigens, such as proteins or peptides, polysaccharides and, more rarely, other molecules such as nucleic acids. Specifically, the term antigen would refer herein to amino acid sequences A and B of the invention, as well as to the fusion amino acid sequence of the invention, i.e., both to aggregating proteins and to chaperones.

A preferred embodiment of this aspect of the invention relates to fragments of the antibodies of the invention for being used in the same way and manner as that described for the antibodies of the present invention. Some examples of the fragments of the antibodies of the invention are shown below:
“Fab” fragments resulting from digestion of a whole antibody with papain and comprising only one antigen-binding site and a CL region and CH1,
“F(ab′)2” fragments resulting from digestion of a whole antibody with pepsin and containing two antigen-binding sites,
“Fab″” fragments containing the light-chain constant domain and the first heavy-chain constant domain (CH1) and has only one antigen-binding site. The Fab′ fragments differ from the Fab fragments by the addition of certain residues at the carboxyl terminal end of the heavy-chain domain CH1 including one or more cysteines of the hinge region of the antibody,
“Fv” is the minimum antibody fragment containing a complete antigen-recognition and -binding site. This region consists of a dimer of one heavy- and one light-chain variable domain in close, non-covalent association. In this configuration, the three hypervariable regions (CDRs) of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the capacity to recognize and bind to the antigen, although at a lower affinity than the entire binding site.
Single-chain FV or “scFv” antibody fragments comprise the VL and VH antibody domains, these domains being present in a single polypeptide chain. Preferably, the VL and VH regions are connected by a polypeptide linker which allows scFv to form desired structures for antigen-binding.
“Diabodies” comprise a heavy-chain variable domain (VH) connected to a light-chain variable domain (VL) in the same polypeptide chain (VH-VL) connected by means of a peptide linker which is too short to allow pairing between the two domains on the same chain. This forces pairing with the complementary domains of another chain and promotes the assembly of a dimeric molecule with two functional antigen-binding sites.
“Bispecific antibodies” (BAb) are individual, divalent antibodies (or fragments thereof effective in immunotherapy) having two specific antigen-binding sites for binding to the antigen in a different manner. The two antigen sites can be coupled to one another chemically or by means of genetic engineering methods known in the art.

All these antibody fragments can additionally be modified using conventional techniques known in the art, for example, using deletion/deletions, insertion/insertions, substitution/substitutions, addition/additions of amino acids and/or recombination/recombinations and/or any other modification/modifications (for example, post-translational or chemical modifications, such as glycosylation and phosphorylation) known in the art, alone or in combination.

### Immunotherapeutic Cellular Composition of the Invention

Another aspect of the invention relates to an immunotherapeutic cell composition, hereinafter immunotherapeutic cellular composition of the invention, comprising at least one activated antigen presenting cell (APC), hereinafter activated antigen presenting cell of the invention, in which said APC are obtained from a patient diagnosed with a disease caused by an aggregating protein of the invention and wherein said APC is stimulated by ex vivo exposure to the composition of the invention. In a preferred embodiment of this aspect of the invention the isolated, activated antigen presenting cell or APC is a dendritic cell (DC).

As it is used herein, the term “antigen presenting cells” or “APCs” refers to cells which are capable of activating T cells, and include, but are not limited to, certain macrophages, B cells, and more preferably, dendritic cells (DCs). “Potent antigen presenting cells” are cells which, after being put in contact with an antigen, can activate CD8+ cytotoxic T lymphocytes (naive CTL) in a primary immune response.

“Dendritic cells” or “DOS” are members of a diverse population of morphologically similar cell types found in lymphoid or non-lymphoid tissues.

These cells are characterized by their distinctive morphology and high levels of MHC-class II expression surface. Steinman et al., Ann. Rev. Immunol. 9: 271 (1991). APCs and DCs can be isolated from a series of tissue sources, and suitably from peripheral blood, as described, for example, in WO2004026238. Preferred immunotherapeutic compositions of the present invention use APCs or DCSs which are isolated from a patient diagnosed with a disease caused by an aggregating protein, or preferably with α-synucleinopathy.

APCs and DCs can be isolated by means of routine methodologies which are available in the art. A suitable example of a methodology for isolating DCs is disclosed in U.S. Pat. No. 5,976,546, U.S. Pat. No. 6,080,409, and U.S. Pat. No. 6,210,662.

### Pharmaceutical Composition of the Invention

Another aspect of the invention relates to a pharmaceutical composition, hereinafter pharmaceutical composition of the invention, comprising the composition of the invention, the nucleotide sequence of the invention, the expression vector of the invention, the host cell of the invention, the antibody of the invention, or the immunotherapeutic cellular composition of the invention. In a preferred embodiment of this aspect, the pharmaceutical composition further comprises a pharmaceutically acceptable vehicle. In another preferred embodiment, the pharmaceutical composition further comprises an adjuvant. In another preferred embodiment, the composition of the invention does not comprise an adjuvant.

The term “adjuvant” refers herein to an agent, as long as it does not have an antigenic effect itself, which can stimulate the immune system increasing its response to the vaccine. Although not being limited thereto, aluminum salts “aluminum phosphate” and “aluminum hydroxide” are the two most commonly used adjuvants in vaccines. Other substances, such as, for example, squalene can also be used as adjuvants.

Another aspect of the invention relates to a dosage form, hereinafter dosage form of the invention, comprising the composition of the invention.

The pharmaceutical compositions provided herein contain therapeutically effective amounts of each of the sequences A and B provided herein which are useful in the treatment of or in improving one or more of the symptoms of aggregating protein toxicity-associated diseases or disorders.

Suitable pharmaceutical vehicles for the administration of the compositions provided herein include any of the vehicles known by the persons skilled in the art which are considered suitable for the particular mode of administration.

Furthermore, the compositions of the invention can be combined with other active ingredients.

The pharmaceutical compositions of the invention can be formulated in suitable pharmaceutical preparations such as solutions, suspensions, tablets, dispersible tablets, pills, capsules, powders or sustained release formulations or elixirs, for oral administration, or in sterile solutions or suspensions for parenteral administration, as well as in preparations of transdermal patches and dry powder inhalers. In a preferred embodiment, the pharmaceutical compositions of the invention are formulated using techniques and methods well known in this field (see, for example, Ansel Introduction to Pharmaceutical Dosage Forms, Fourth Edition 1985, 126).

The active ingredients can be administered at one go, or can be divided into several smaller doses to be administered at time intervals. It is understood that the dosage and the precise duration of treatment depend on the disease being treated and can be determined empirically using known test protocols or by extrapolation from in vivo or in vitro assay data. It must be indicated that the concentrations and values of dosage also can change with the severity of the condition to be alleviated. Furthermore, it must be understood that for any particular subject, the specific dosage regimens must be adjusted over time according to individual need and the professional criterion of the person administering or supervising the administration of the compositions, and that the concentration ranges described herein are merely illustrative and do not seek to limit the scope or practice of the claimed compositions.

As it is used herein, the term “active ingredient”, “active substance”, “pharmaceutically active substance”, or “pharmaceutically active ingredient” means any component which potentially provides a pharmacological activity or another different effect in diagnosing, curing, mitigating, treating or preventing a disease, or which affects the structure or function of the human body or the body of other animals. The term includes those components promoting a chemical change in the preparation of a drug and are present therein in an envisaged modified form providing the specific activity or the effect. In the present invention it primarily refers, but is not limited, to the combination of sequences A and B of the invention, as well as to the fusion amino acid sequence of the invention, the antibodies of the invention and the activated antigen presenting cell of the invention.

In another preferred embodiment of the invention, the pharmaceutical composition of the invention is a vaccine.

### Combined Preparation of the Invention

Sequences A and B of the invention can be found bound to one another in one and the same composition, or juxtaposed for simultaneous, combined or sequential administration to a mammal, including human beings.

Therefore, another aspect of the invention relates to a combined preparation, hereinafter combined preparation of the invention, comprising sequences A and B of the invention or any of their biologically active fragments and/or variables.

It must be emphasized that the term “combined preparation”, or also referred to as “juxtaposition”, herein means that the components of the combined preparation do not need to be bound to one another, for example, in a true composition, so that they will be available for combined, separate or sequential application. The expression “juxtaposed” therefore means that a true combination is not necessary in view of the physical separation of the components.

In another preferred embodiment of the invention, the combined preparation of the invention is a vaccine.

### Uses of the Invention

Another aspect of the invention relates to the use of sequences A and B of the invention, the composition of the invention, the nucleotide sequence of the invention, the expression vector of the invention, the host cell of the invention, the antibody of the invention, the immunotherapeutic cellular composition of the invention, the pharmaceutical composition of the invention, or the combined preparation of the invention, in the preparation of a medicinal product. In a preferred embodiment, the medicinal product is a vaccine. Therefore, another aspect of the invention relates to a vaccine, hereinafter vaccine of the invention, comprising sequences A and B of the invention, the composition of the invention, the nucleotide sequence of the invention, the expression vector of the invention, the host cell of the invention, the antibody of the invention, the immunotherapeutic cellular composition of the invention, the pharmaceutical composition of the invention, or the combined preparation of the invention.

In the context of the present invention, the term “vaccine” refers to an antigenic preparation used for establishing the response of the immune system to a disease. They are antigen preparations which cause, once in the body, the immune system response, by means of antibody production, and generate immunological memory producing permanent or temporary immunity.

The vaccine can contain a single type of peptide or a variety of peptides covering different or similar epitopes. Furthermore, or as an alternative, a single polypeptide with multiples epitopes can be provided. This last type of vaccine is referred to polyvalent vaccine. A multiple epitope includes two or more repetitive epitopes.

Another aspect of the invention relates to the use of sequences A and B of the invention, the composition of the invention, the nucleotide sequence of the invention, the expression vector of the invention, the host cell of the invention, the antibody of the invention, the immunotherapeutic cellular composition of the invention, the pharmaceutical composition of the invention, or the combined preparation of the invention, in the preparation of a medicinal product for the treatment or prevention of aggregating protein-related diseases or disorders.

In a preferred embodiment of this aspect of the invention, the aggregating protein is α-synuclein or α-synucleinopathies. In a preferred embodiment of this aspect, the α-synuclein-related disease or disorder is selected from the list consisting of:

Parkinson's disease (PD), Lewy body dementia (LBD), the Lewy body variant of Alzheimer's disease, multiple system atrophy (MSA), Alzheimer's disease, Down syndrome, amyotrophic lateral sclerosis (ALS), frontotemporal dementia, Gaucher disease, Huntington's disease, prion disease, Creutzfeldt-Jakob disease, multiple sclerosis, Gerstmann-Sträussler-Scheinker syndrome, Kuru, fatal familial insomnia, cerebrovascular amyloidosis, glaucoma, age-related macular degeneration, neurodegeneration due to age-related protein aggregation, psychiatric syndromes, schizophrenia and/or schizophrenia-like disorders. More preferably, it is Parkinson's disease (PD).

In another preferred embodiment, the aggregating protein is SOD1, and even more preferably the disease is amyotrophic lateral sclerosis (ALS).

Another aspect of the invention relates to the use of the combined preparation of the invention, comprising sequences A and B of the invention, in the preparation of a medicinal product for combined, simultaneous or sequential administration, for the treatment or prevention of aggregating protein-related diseases or disorders of the invention. In a preferred embodiment, the aggregating protein is α-synuclein or synucleinopathies.

As it is used herein, the term “medicinal product” refers to any substance used for preventing, diagnosis, alleviating, treating or curing diseases in human beings and animals. In the context of the present invention it also refers to a composition capable of generating an immune response against an aggregating protein given that it is causing a disease in human beings or animals. It therefore includes what is known as vaccine, as defined above in this specification.

### Diagnostic Method of the Invention

Another aspect of the invention relates to a method for the in vitro detection of an aggregating protein of the invention which comprises adding the antibody of the invention to a biological sample comprising or suspected of comprising said aggregating protein, and detecting and measuring a concentration of any complex formed between the antibody and the aggregating protein. In a preferred embodiment, said aggregating protein is α-synuclein. More preferably, the antibody is labeled with a detectable marker, and the presence of the complex formed between the antibody and α-synuclein is highlighted through detection of the marker. In another preferred embodiment, detection of the aggregating protein can be used for the diagnosis, prognosis or follow-up of a disease related with said aggregating protein. Said proteins and diseases have been described above in a non-limiting manner in the specification.

The terms “polynucleotide” and “nucleic acid” are used herein interchangeably, referring to polymeric forms of nucleotides of any length, both ribonucleotides (RNA) and deoxyribonucleotides (DNA).

The terms “amino acid sequence”, “peptide”, “oligopeptide”, “polypeptide” and “protein” are used herein interchangeably, and refer to a polymeric form of amino acids of any length, which can be chemically or biochemically modified, coding or non-coding forms.

Throughout the description and the claims the word “comprises” and variants thereof do not intend to exclude other technical features, supplements, components or steps. For persons skilled in the art, other objects, advantages and features of the invention will be inferred in part from the description and in part from the practice of the invention. The following examples and drawings are provided by way of illustration and they are not meant to limit the present invention.

### EXAMPLES OF THE INVENTION

Overexpression and Purification of α-Synuclein

Human wild-type α-synuclein (αSyn) was overexpressed in E. coli BL21 (DE3) cells using the plasmid pT7-7 and purified as previously described (Roodveldt et al., 2010. PLoS One 5(10): e13481; Roodveldt et al., 2013. PLoS One. 8(11):e79160). The purity (>95%) and monomeric state of the preparation was evaluated using 15% SDS-PAGE, mass spectrometry, native electrophoresis in 4-10% gradient (Lonza, Basel, Switzerland), as described (Roodveldt et al., PLoS One 2010). Before use and for the elimination of any possible endotoxin content, the protein in solution was subjected to filtration through centrifugation with Amicon Ultra-100 kDa (Merck Millipore Ltd., Carrigtwohill, IRL). The endotoxin content in the protein preparation was measured with the Oxisensor Chromogenic LAL Assay Kit (GenScript, Piscataway, U.S.A.), and the values obtained were <1 EU/mg protein. The αSyn protein concentration was determined with Micro BCA Reagent Kit (Pierce, Rockford, Ill., U.S.A.).

Preparation and Characterization of α-Synuclein Oligomers

The preparation and characterization of αSyn soluble oligomers was carried out as described (Roodveldt et al., 2012. Biochemistry 51(44):8771-8) and the purified oligomeric fraction was stored at 4° C. up to a maximum of 24 hrs. The protein concentration was determined as described above.

Grp94 Protein

Canine full-length recombinant Grp94 (Gp96) expressed in baculovirus (endotoxin-free) from Abcam (Cambridge, United Kingdom; #ab92290) was used.

Hsp70 Protein

Full-length human cytosolic recombinant Hsp70 (HSPA1A) protein overexpressed in E. coli and purified as described Was used (Roodveldt et al., 2009. EMBO J. 28(23):3758-70). The purity (>95%) of the preparation was evaluated using 15% SDS-PAGE. Before use and for the elimination of any possible endotoxin content, the protein in solution was subjected to filtration through centrifugation with Amicon Ultra-100 kDa (Merck Millipore Ltd., Carrigtwohill, Ireland). The endotoxin content in the protein preparation was measured with the Oxisensor Chromogenic LAL Assay Kit (GenScript, Piscataway, U.S.A.), and the values obtained were <1 EU/mg protein. The αSyn protein concentration was determined with Micro BCA Reagent Kit (Pierce, Rockford, Ill., U.S.A.).

SOD1 Protein

Full-length human recombinant superoxide dismutase 1 (SOD1) protein overexpressed in E. coli purified by chromatography and with a purity >95% (ProSpec, Rehovot, Israel) was used.

Mouse Immunization Protocols

Protocol 1:

The mice were immunized with 5 μg of Grp94 and/or with 0.8 μg of non-aggregated αSyn. Preparation of protein solution for immunization: after incubating Grp94 and αSyn with a chaperone:αSyn molar ratio of 1:1 (in 20 mM HEPES, pH 7.5) in combination or alone (in the cases of controls) for 2 hours at 42° C., the solution was brought to a final volume of 150 μl of DPBS per mouse. It was then mixed with another 150 μl of complete Freund's adjuvant and the mixture was stirred vigorously until obtaining an emulsion. Each mouse was administered 300 μl of emulsion, distributed in three subcutaneous injections: one in the upper part of each rear paw and another in the cervical area. The same process was performed after 7 days but in this case using incomplete Freund's adjuvant. The mice were sacrificed 7 days after the second immunization.

Protocol 2:

The mice were inoculated with 8 μg of Grp94 and/or 13 μg of αSyn oligomers, Preparation of protein solution for immunization: after incubating Grp94 and oligomeric αSyn with a chaperone:αSyn protomer molar ratio of 1:10 (in 20 mM HEPES, pH 7.5) in combination or alone (in the cases of controls) for 2 hours at 42° C., the solution was brought to the total volume of 100 μl per mouse. This solution, in the absence of adjuvant, was inoculated in a single subcutaneous injection applied in the lumbar area. The same process was repeated after 7 days and 21 days following the first immunization. Seven days after the last immunization, i.e., 28 days after the start of the process, the animals were sacrificed.

In both cases, between 500 and 700 μl of blood were drawn from the mice to obtain serum and analyze the presence of antibodies, and the spleen was extracted for extracting the splenocytes which were used for analyzing the regulatory T lymphocyte (Treg) population and the immune response against αSyn.

Protocol 3:

The mice were immunized with a combination of 11 μg of Hsp70 and 2.5 μg of non-aggregated SOD1, or alternatively with 11 μg of Hsp70, or 2.5 μg of SOD1, or a vehicle, as controls. Preparation of protein solution for immunization: after incubating Hsp70 and SOD1 with a chaperone:SOD1 molar ratio of 1:1 (in 20 mM Tris, pH 7.4) in combination or alone (in the cases of controls) for 2 hours at room temperature, the solution was brought to a final volume of 150 μl of PBS per mouse. It was then mixed with another 150 μl of complete Freund's adjuvant and the mixture was emulsified transferring the mixture between two syringes for 5 minutes. Each mouse was administered 300 μl of emulsion, distributed in three subcutaneous injections: one in the upper part of each rear paw and another in the cervical area. The same process but in this case using incomplete Freund's adjuvant was performed after 14 and 28 days following the first injection in order to enhance the response.

Protocol 4: Immunization With αSyn and Hsp70 Without Adjuvant (Manuscript)

The mice were immunized with 5 μg or 50 μg of Hsp70 (low and high doses, respectively) and/or with 1.06 μg or 10.6 μg of non-aggregated αSyn (low and high doses, respectively). Preparation of protein solution for immunization: the proteins Hsp70 and αSyn were incubated with a chaperone:αSyn molar ratio of 1:1 (Tris/HCl 50 mM, pH 7.4; 150 mM KCl, 2 mM MgCl₂) in combination or alone (in the cases of controls) at 37° C. for 2 hours in the presence of 4 mM ATP after which 2.5 mM ADP was added and incubated 2 more hours. The solution was brought to a final volume of 200 μl of PBS per mouse and the mice were administered a single subcutaneous injection in the lumbar area. The same process was performed after 7 days. The mice were sacrificed 7 days after the second immunization.

Adoptive Splenocytes Transfer

After the immunization process according to protocols 1 and 2 and sacrificing the immunized mice, an equal number of splenocytes originating from each mouse of one and the same group was mixed and 10 million splenocytes were transferred to each recipient mouse by means of a single intraperitoneal injection. After the transfer, the mice were given a leeway of 10 days before starting the treatment with MPTP in order to allow the splenocytes to establish themselves in the host.

Treatment of Mice With MPTP

The animals receiving the adoptive transfer were chronically treated with MPTP as described above (Villadiego J. et al., 2005. 25, 4091-4098). After treatment, the animals were left to recover for one month before proceeding with the behavioral tests.

Behavioral Evaluations of the Animals Treated With MPTP

Behavioral analysis (footprinting) was carried out after leaving the mice to recover for one month after chronic treatment with MPTP and amphetamines were then administered following the protocol previously described in Muñoz-Manchado A. B. et al, 2013. Neurobiol Aging 34(3):902-15.

Sacrificing Treated Animals and Extraction of Samples

After treatment with MPTP and subsequent recovery and behavioral evaluation, the mice were left for 10 days to assure the complete elimination of amphetamines and to dismiss any undesired interaction. The mice were sacrificed and their blood was drawn to obtain serum, and the spleen was extracted for isolating splenocytes.

Evaluation of the Immune Response in Immunized and Recipient Animals

Determination of Treg Cells Using Flow Cytometry:

Splenocytes were isolated from the spleen using a standard protocol by means of perfusion described above. 10⁶ cells were labeled following the manufacturer's recommendations using the Mouse Foxp3 Buffer Set (BD Pharmigen™) and anti-CD4 FITC, anti-CD25 APC and anti-Foxp3 PE antibodies, all from BD Pharmigen™. They were analyzed using a FACS Calibur flow cytometer and the Cell Quest Pro software (BD Pharmigen™)

In Vitro Stimulation With Antigen and Determination of Cytokines (IFN-γ, IL-10 and IL-17):

3×10⁶ cells were divided into three wells and stimulated, depending on the case, with culture medium alone, αSyn (15 μg per well), SOD1 (20 μg per well), or LPS (0.5 μg per well) in 1 ml of culture medium. After 36 hours, the supernatants were collected and centrifuged to eliminate cell debris and cells and kept at −80° C. for subsequent cytokine analysis.

To measure IFN-γ, IL-10 and IL-17, specific ELISA kits, BD OptEIA™ Set Mouse IFN-gamma, BD OptEIA™ Set Mouse IL-10 (BD Pharmigen™) and BD OptEIA™ Set Mouse IL17(BD Pharmigen™) were used and ELISAs were performed following the manufacturer's instructions.

Determination of Total IgG and Anti-αSyn or Anti-SOD1 in Serum:

The blood drawn while sacrificing the animals was left at room temperature for one hour and at 4° C. for another hour, thereby allowing it to clot. It was then centrifuged at 21,000 g for 15 minutes in order to obtain cell-free serum which was frozen at −80° C. for subsequent analysis. To measure the IgG1 and IgG2a levels as well as total IgM and IgG levels, the sera were diluted 1:240000 in PBS, 100 μl per well were transferred to a MaxiSorp 96-well plate (NUNC) and incubated for 1 hour at 37° C., they were then washed with PBST (0.05% Tween 20), blocked for 1 hour at 37° C. with Assay Diluent (BD Pharmigen™). After washing the wells again, a specific anti-IgG antibody (Promega), IgG1 (AbCam), IgG2a (AbCAm) or IgM (Miltenyi) conjugated to a HRP diluted 1:4000 in Assay Diluent was used and incubated for 1 hour at 37° C. After washing the wells, TMB Substrate Reagent (BD Pharmigen™) was used to determine the amount of IgG, IgG1, IgG2a or IgM present in the sera.

A similar protocol but with variations was used to measure the amount of specific anti-αSyn antibodies. Briefly, the plate was pretreated either with αSyn diluted in PBS or an anti-αSyn antibody (N19, Santa Cruz) at 1 μg per well in 100 μl and incubated 2 hours at 37° C., the wells were then washed and blocked like in the preceding protocol. In the case of having pretreated with anti-αSyn antibody, the wells were incubated with 10 μg per well of αSyn for 1 hour at 37° C. and washed with PBST. In both cases, 100 μl per well were incubated with sera diluted 1:50 in Assay Diluent for an hour at 37° C., then washing, detection and development were carried out in the same manner as in the preceding protocol.

A similar protocol but with variations was used to measure the amount of specific anti-SOD1 antibodies. Briefly, the plate was pretreated with SOD1 diluted in PBS at 0.5 μg per well in 100 μl and incubated 2 hours at 37° C., the wells were then washed and blocked like in the preceding protocol and 100 μl per well were incubated with sera diluted 1:50 in Assay Diluent for an hour at 37° C., then washing, detection and development were carried out in the same manner as in the preceding protocol.

Results

In Vitro Stimulation of Splenocytes With a Grp94/Gp96 and α-Synuclein Combination to Evaluate Their Immune Response-Inducing Potential

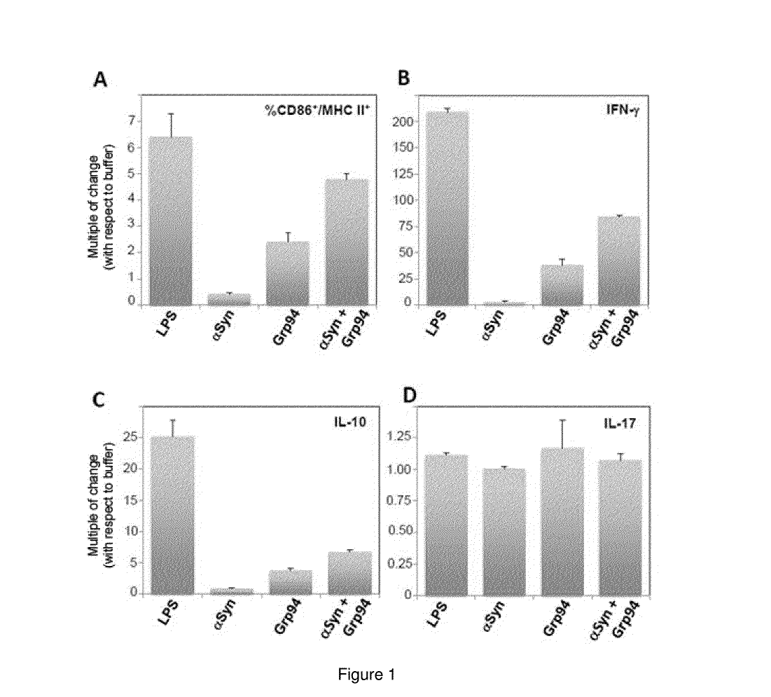

In order to promote an immune response against α-synuclein (αSyn) which may potentially act as a neuroprotector in the context of a synucleinopathy, the effect of an αSyn and Grp94 combination was first evaluated in vitro. To that end, a culture of murine splenocytes with a Grp94 and αSyn combination (1:1 molar ratio), or the equivalent amounts of Grp94 or αSyn as controls, were incubated for 24 hrs. After separating the cells, they were analyzed using flow cytometry quantifying the expression of cell activation surface markers, particularly MHC II and CD86, observing a 50% reduction and a 2-fold increase in the expression of these markers in the case of stimulation with αSyn and Grp94, respectively, with respect to the stimulation control with buffer solution (FIG. 1A). On the other hand, the stimulation of splenocytes with an αSyn and Grp94 combination (αSyn+Grp94) caused a 4.5-fold increase in the expression of these markers with respect to the control (FIG. 1A).Furthermore, the levels of cytokines IFN-γ, IL-10, and IL-17 were measured in the supernatants using ELISA (FIGS. 1B, 1C, 1D), also detecting relative increases for IFN-γ and IL-10 in the case of stimulation with the combination of proteins (FIGS. 1B and 1C).

Immunization of Mice With a Grp94/Gp96 and α-Synuclein Combination, Adoptive Transfer of Their Splenocytes to Recipient Mice and Subsequent Administration of MPTP (Model of Parkinson's Disease) to Evaluate its PD Protection Potential

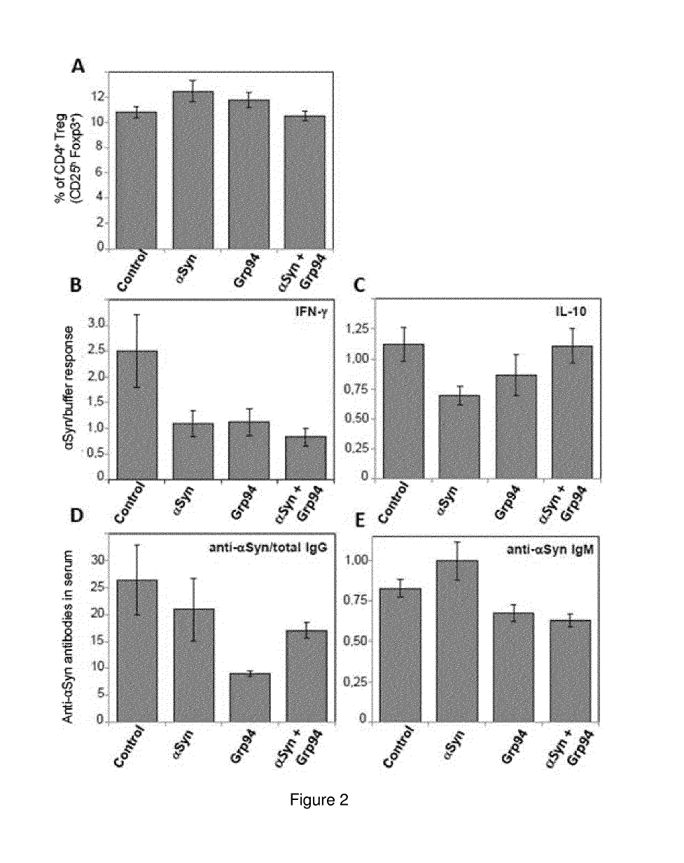

Having observed a different response of the splenocytes in culture as a result of stimulation with the combination of proteins, healthy C57BL/6 male mice 5-7 weeks of age (5 mice per group) were immunized with said preparations (αSyn, Grp94, an αSyn and Grp94 combination, and buffer as control) to evaluate the in vivo response. To that end, two vaccination protocols were designed (protocol 1 and 2). Seven days after finish applying immunization protocol 1, the mice were sacrificed for analyzing the splenocytes and serum. First, no changes were observed in the regulatory T-cell (Treg) content in relation to the total CD4⁺ cells (FIG. 2A). Next, the splenocytes were cultured and alternatively incubated with αSyn or with buffer solution as a reference, after which the supernatants were separated for analyzing the levels of certain cytokines (FIGS. 2B and 2C). In this case, a different response was also observed for the αSyn and Grp94 combination, proving a minimum reduction in the levels of IFN-γ (FIG. 2B) and equivalent values of IL-10 (FIG. 2C) for αSyn+Grp94, as a result of the in vitro stimulation of splenocytes with αSyn. Finally, the levels and proportion of anti-αSyn antibodies in serum were measured. First, and surprisingly, a significant reduction in the anti-αSyn IgG antibody content was observed in the case of mice immunized with αSyn alone (FIG. 2D), whereas immunization with αSyn+Grp94 produced intermediate levels of anti-αSyn IgG antibodies in relation to immunizations with αSyn and Grp94 (FIG. 2D).

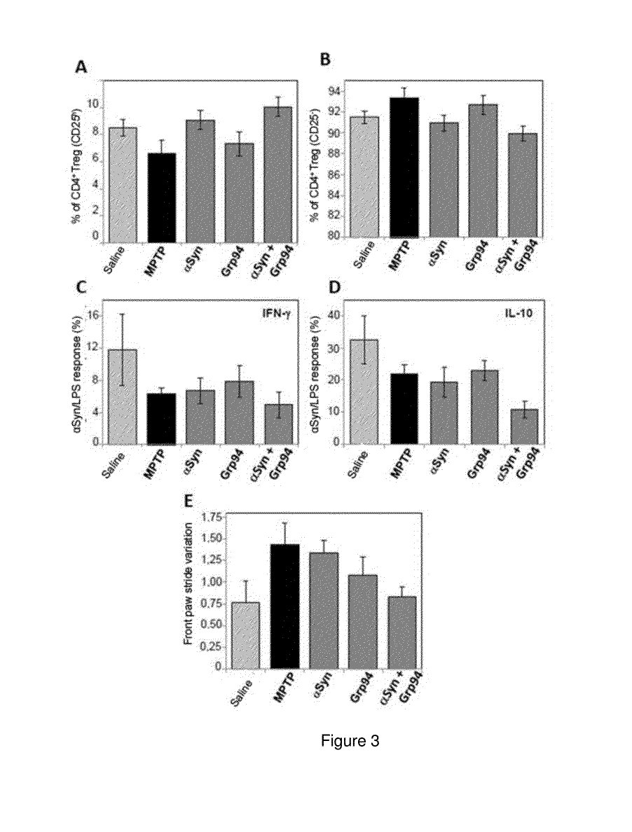

On the other hand, as a strategy to evaluate the synucleinopathy therapeutic potential, splenocytes originating from the mice immunized according to vaccination protocol 1 were transferred to healthy C57BL/6 mice, chronic treatment with MPTP was applied to such mice for 3 months as a model of Parkinson's disease (see Materials and Methods). Forty-three days after the administration of the last dose of MPTP for all the groups (except for the ‘saline control’) and after having performed the behavioral tests, the mice were sacrificed and the spleens and serum extracted for analysis. The isolated splenocytes were analyzed using flow cytometry and cultured and stimulated in vitro with αSyn or buffer for analyzing the cytokine-mediated response (FIG. 3 A-D). According to the results, the animals treated with MPTP which have not received splenocytes (MPTP control) had lower levels of Treg cells than animals not treated with MPTP or with splenocytes (‘healthy’ or ‘saline’ control) (FIG. 3A). The recipient animals treated with splenocytes originating from animals immunized with αSyn or Grp94 showed values somewhat greater and less than ‘saline’, respectively, whereas the recipients of splenocytes from mice immunized with αSyn+Grp94 showed the highest percentages of Treg (FIG. 3A). The same but opposite profile was observed for effector T cells (Teff, FIG. 3B), with coinciding levels for ‘αSyn+Grp94’ and ‘saline’ mice.

On the other hand, the response of the cultured splenocytes with respect to the stimulation with αSyn or LPS was evaluated in vitro. In this case, a reduction was observed in the supernatant levels of IFN-γ and IL-10 for the splenocytes from the recipients of cells of animals immunized with αSyn+Grp94, in relation to the equivalents of αSyn and Grp94 alone (FIGS. 3B and 3C), demonstrating a different effect of vaccination with the αSyn and Grp94 combination.

Finally, the results of the behavioral test (footprinting) in recipient animals showed minimum and maximum values of the measured parameter (stride variation) of average ±S.E.M. of 1.43±0.25 (N=5) and 0.76±0.26 (N=4) for ‘MPTP’ and ‘saline’ controls, respectively, and a value close to the ‘healthy’ control of 0.83±0.17 (N=7) for treatment with ‘αSyn+Grp94’, less than that obtained with treatment with ‘αSyn’ or ‘Grp94’ alone (FIG. 3E).

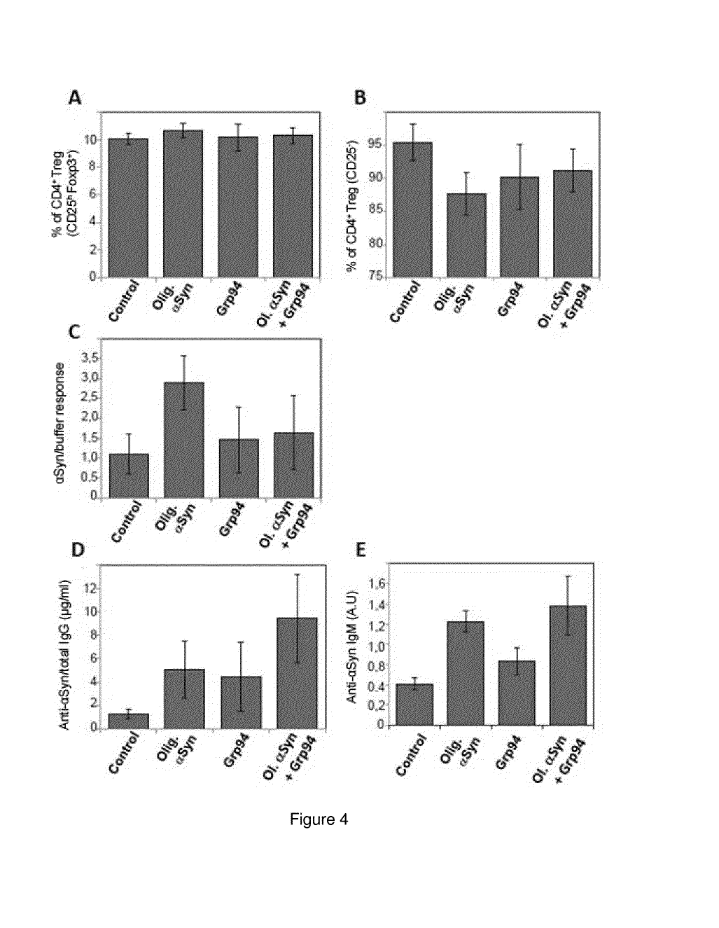

A second immunization protocol (protocol 2) was then tested in healthy C57BL/6 male mice 5-7 weeks of age (5 mice per group), in which 3 immunizations were performed in the absence of adjuvant, and oligomeric/aggregated αSyn was used. In this case, no differences were observed in the Treg content with respect to the total CD4⁺ cells (FIG. 4A), although a certain tendency towards a higher Teff cell content in mice immunized with ‘Ol. αSyn+Grp94’ with respect to those immunized with Ol. αSyn and Grp94 separately was detected (FIG. 4B). Like before, the levels of IFN-γ in the supernatants of splenocytes cultured and stimulated in vitro with αSyn or buffer solution as a reference were measured, observing an increase in the samples originating from immunization with Ol. αSyn, and values similar to the control for ‘Grp94’ and ‘Ol. αSyn+Grp94’ (FIG. 4C). Furthermore, the results of serum anti-αSyn IgG antibody content in relation to total IgG, and of anti-αSyn IgM antibody content showed higher levels in the case of mice immunized with ‘Ol. αSyn+Grp94’ than those immunized with Ol. αSyn or Grp94 separately (FIGS. 4D and 4E).

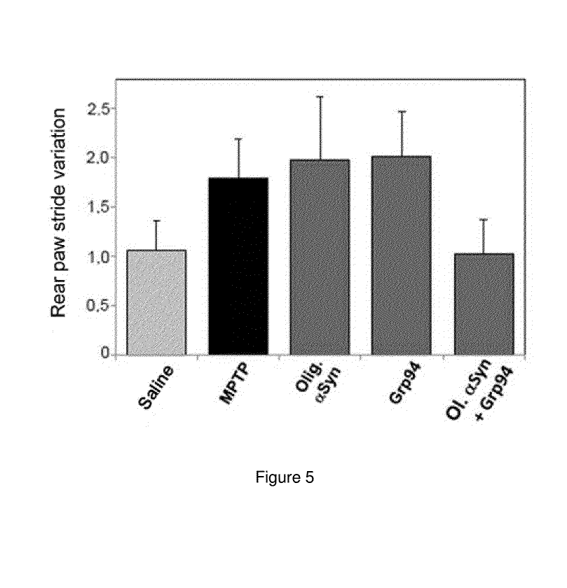

Like before, splenocytes from the mice immunized according to protocol 2 were transferred to healthy male C57BL/6 mice, MPTP was then administered to said mice for 3 months as a model of Parkinson's diseases. The results of the behavioral test (footprinting) in the recipient animals (FIG. 5) showed minimum and maximum values of the measured parameter (stride variation) of average ±S.E.M. of 1.79±0.26 (N=5) and 1.06±0.16 (N=5) for ‘MPTP’ and ‘saline’ controls, respectively, and a value similar to that of the ‘healthy’ control of 1.02±0.23 (N=5) for treatment with ‘αSyn+Grp94’, differing from that obtained with the treatment with ‘αSyn’ or ‘Grp94’ alone (1.98±0.39, N=5; 2.01±0.55, N=5) (FIG. 5).

These results demonstrate that immunization with a combination of αSyn (both in the non-aggregated and oligomeric forms) and the chaperone Grp94 produces different effects on the immune response of the immunized mice and that the adoptive transfer of splenocytes originating from said immunization is capable of preventing or counteracting the development of symptoms related to Parkinson's disease in the recipient mice.

Vaccination of a Transgenic Mice Model of ALS (SOD1 G93A) With an Hsp70 and SOD1 Combination to Evaluate Their ALS Therapeutic/Prophylactic Capacity

A preliminary experiment was conducted to evaluate the therapeutic potential of immunization with a combination of SOD1 and the chaperone Hsp70. To that end, a transgenic mice model of ALS (Tg SOD1 G93A, Jackson Laboratories Inc., U.S.A.) was used with mice 9 weeks of age (3 mice per group; N=3) that were immunized according to protocol 3. The mice were then carefully monitored by means of measuring various standard parameters of murine models of ALS, starting before the onset of the disease (typically in week 12-13), and until the termination of the disease according to the ‘end point’ criterion (typically in week 18-19, when the animal's weight drops up to <80% with respect to its maximum weight). After being sacrificed at the ‘end point’, the spleen and blood were extracted for analyzing the splenocytes and serum.

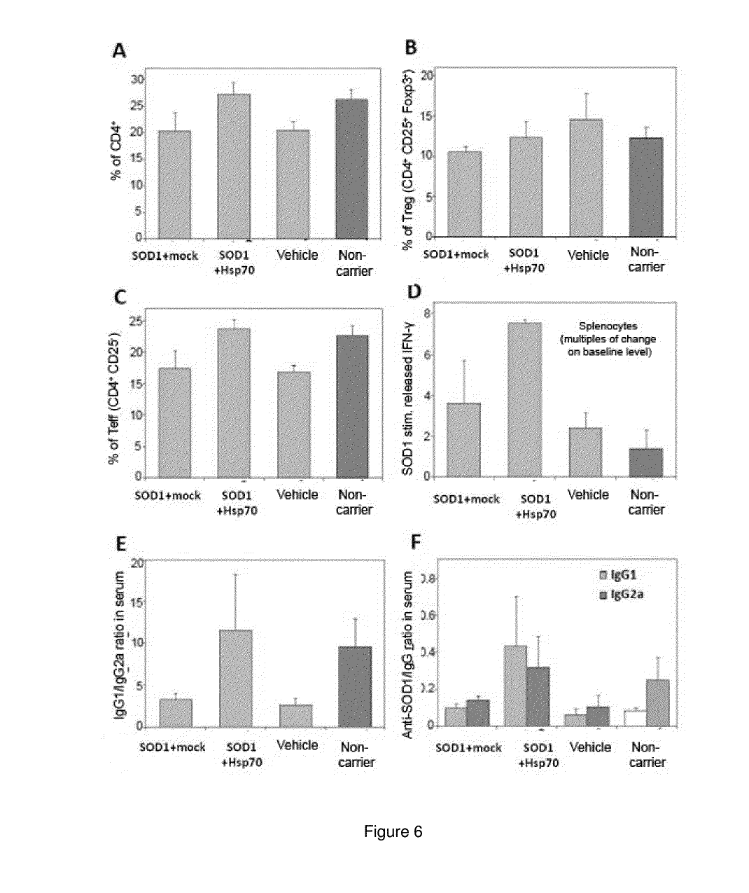

The isolated splenocytes were analyzed using flow cytometry to analyze the CD4⁺ cell, regulatory T cell (Treg) and effector T cell (Teff) populations (FIG. 6).

According to this analysis, immunization with SOD1+Hsp70 produces an increase in the CD4⁺ population and the Teff cell content with respect to animals treated with a vehicle, whereas no changes are observed in the Treg cell content. On the other hand, the isolated splenocytes were stimulated in vitro with SOD1 for quantifying IFN-γ secretion, as an indicator of the type of antigen-specific response (FIG. 6). The data obtained shows a 3-fold increase of the levels of IFN-γ secreted upon stimulation with SOD1 with respect to the control. Finally, the levels of IgG antibodies in serum were measured to analyze the IgG1/IgG2a ratio in the different groups. The results of this quantification indicate an increase of said quotient in the case of the SOD1+Hsp70 group both for total IgG antibodies, and for the SOD1-specific antibody population (FIG. 6), suggesting the importance of Th2-type or ‘neuroprotective’ response in the context of neurodegenerative diseases.

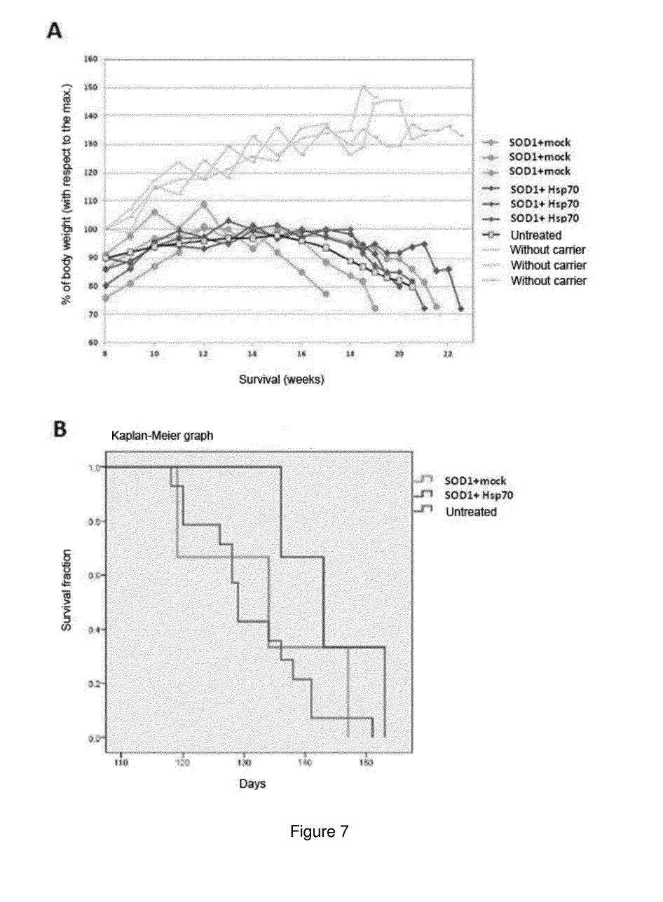

On the other hand, analysis of the weight evolution and survival of each mouse allows observing a certain protection against weight loss in mice immunized with SOD1+Hsp70 with respect to controls (FIG. 7), as well as an increase in survival (FIG. 7). These preliminary results indicate a different immune response induced by SOD1+Hsp70 and a potential beneficial effect of this vaccination strategy against ALS.

Immunization of C57BL/6 Mice With an Adjuvant-Free αSyn and Hsp70 Complex/Combination and Characterization of the Immune Response Generated

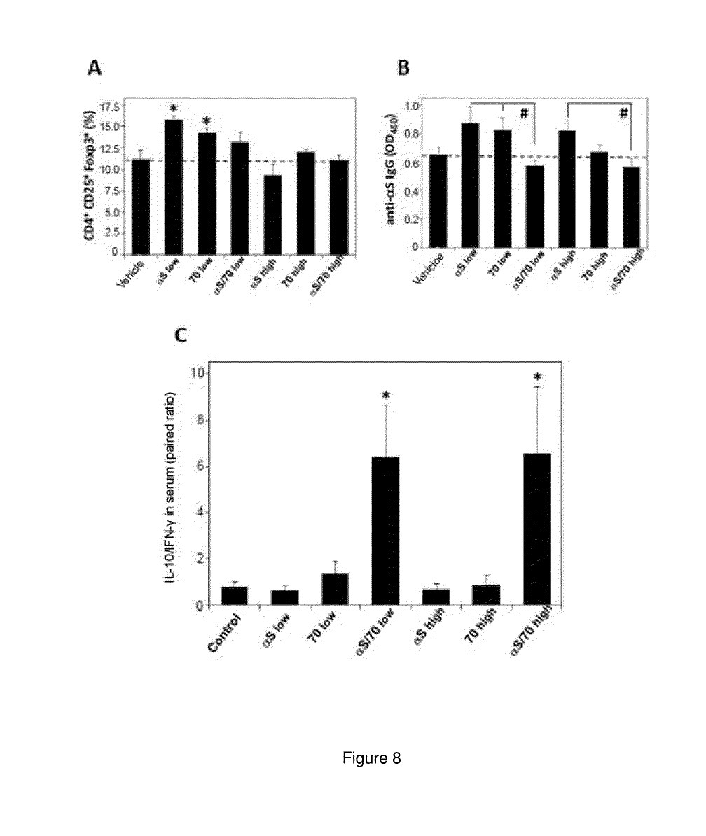

First, the formation of a complex between αSyn and Hsp70 was demonstrated by means of PAGE-Western blot and Biacore after pre-incubating the proteins in buffer. Healthy C57BL/6 mice (N=6-7) were immunized with two different doses ('low' and ‘high’ doses) of said preparation or with the corresponding controls prepared in the same manner, in the absence of adjuvant, according to protocol 4. Seven days after the end of the immunization protocol, the mice were sacrificed and the spleen and blood were extracted for isolating splenocytes and obtaining serum. The analysis of splenocytes using flow cytometry showed that, while immunization with αSyn or Hsp70 alone causes an increase in the Treg population, the αSyn+Hsp70 group is capable of suppressing said effect, keeping that population within the ‘normal’ levels with respect to the control (FIG. 8). On the other hand, a similar different effect for αSyn+Hsp70 was observed in the levels of anti-αSyn IgG antibodies, indicating that the response induced by the complex may be capable of suppressing an antigen-specific humoral response for the autoantigen αSyn (FIG. 8). Finally, the levels of IL-10 and IFN-γ in serum were quantified, and the IL-10/IFN-γ paired quotients for each mouse (FIG. 8) were analyzed. The results obtained clearly demonstrate a different increase of said quotient for animals immunized with the αSyn+Hsp70 combination, indicating that the response induced is a Th2-type or ‘neuroprotective’ response in the context of neurodegenerative diseases.

1. A composition comprising:
a) a mixture of peptides/polypeptides/proteins comprising i) at least one peptide/polypeptide/protein the amino acid sequence of which corresponds with an aggregating peptide/polypeptide/protein (“sequence A of the invention”) or with any of the biologically active variants and fragments of said sequence, and ii) a peptide/polypeptide/protein the amino acid sequence of which corresponds with a molecular chaperone (“sequence B of the invention”) or with any of the biologically active variants and fragments of said sequence; and/or
b) a fusion peptide/polypeptide/protein, “fusion amino acid sequence of the invention”, comprising i) the amino acid sequences of at least one aggregating peptide/polypeptide/protein (“sequence A of the invention) or any of the biologically active variants and fragments thereof, and ii) the sequences of at least one peptide/polypeptide/protein the amino acid sequence of which corresponds with a chaperone (“sequence B of the invention”) or with any of the biologically active variants and fragments thereof.

2. The composition according to the preceding claim, where the aggregating peptide/polypeptide/protein is selected from the list consisting of α-synuclein, huntingtin, beta-amyloid peptide, TDP-43, FUS, tau, prion protein, fibronectin and SOD1.

3. The composition according to any of claims 1-2, where the aggregating peptide/polypeptide/protein is α-synuclein or any of the biologically active variants and fragments thereof.

4. The composition according to any of claims 1-3, where the chaperone is selected from the list consisting of the Hsp90 family, Hsp70 family, immunophilin family, peptidase C56 family, PPlase family, 14-3-3 family, small heat-shock protein group, small GTPase family, Hsp40 (DnaJ) family, and clusterin, Grp170, calreticulin, Hsp105, CHIP, alpha-crystallin or any of the combinations thereof.

5. The composition according to any of claims 1-4, where the chaperone belongs to the Hsp90 family and is selected from the list consisting of Grp94, Grp96 and Hsp90 or any of the combinations thereof.

6. The composition according to any of claims 1-4, where the chaperone belongs to the Hsp70 family and is selected from the list consisting of Hsp70, Hsc70 and Grp75 or any of the combinations thereof.

7. The composition according to any of claims 1-4, where the chaperone belongs to the immunophilin family and is selected from the list consisting of FKBP12 and FKBP4/FKBP52 or the combination thereof.

8. The composition according to any of claims 1-4, where the chaperone is DJ1 /PARK7.

9. The composition according to any of claims 1-4, where the chaperone belongs to the PPlase family and is selected from the list consisting of Pin1, CypA, Cyp40 or any of the combinations thereof.

10. The composition according to any of claims 1-4, where the chaperone belongs to the 14-3-3 family and is selected from the list consisting of 14-3-3 gamma, 14-3-3 epsilon, 14-3-3 tau or any of the combinations thereof.

11. The composition according to any of claims 1-4, where the chaperone is Hsp27.

12. The composition according to any of claims 1-4, where the chaperone is Rab11A.

13. The composition according to any of claims 1-4, where the chaperone of the Hsp40 (DnaJ) family is selected from Hsp40 and CSP or the combination thereof.

14. The composition according to any of claims 1-4, where the chaperone is selected from the list consisting of clusterin, Grp170, calreticulin, Hsp105, CHIP, alpha-crystallin or any of the combinations thereof.

15. The composition according to claim 1, where the aggregating peptide/polypeptide/protein is α-synuclein or SOD1 and the chaperone is selected from any of those defined in claims 4-14.

16. The composition according to any of claims 1-4, where the chaperone is Grp94 or any of its biologically active variants, and combinations thereof.

17. The composition according to claim 1, where the aggregating peptide/polypeptide/protein is α-synuclein and the chaperone is Grp94, Grp96 or Hsp70, or where the aggregating peptide/polypeptide/protein is SOD1 and the chaperone is Hsp70.

18. The composition according to any of claims 1-17, where at least one of the sequences A and/or B are recombinant sequences.

19. A nucleotide sequence comprising one or more nucleotide sequences encoding the fusion protein as defined in any of claims 1-18.

20. An expression vector comprising the nucleotide sequence of the preceding claim.

21. A host cell comprising the expression vector according to claim 20 or the nucleotide sequence of claim 19.

22. An antibody or a fragment thereof capable of binding to one of sequences A or B, or to the fusion amino acid sequence according to any of the preceding claims.

23. An antibody or a fragment thereof obtained or obtainable after the immunization of an animal, or of the cells of an animal, with the composition as defined in any of claims 1-18.

24. The antibody according to the preceding claim, where the animal used for the immunization is a mammal.

25. An immunotherapeutic cellular composition comprising at least one isolated, activated antigen presenting cell (APC), wherein said APC is obtained from a patient diagnosed with a disease caused by an aggregating protein of the invention, and wherein said APC is stimulated by ex vivo exposure to a composition as defined in any of claims 1-18.

26. The immunotherapeutic cellular composition according to the preceding claim, where the isolated, activated antigen presenting cell or APC is a dendritic cell (DC).

27. A pharmaceutical composition comprising the composition according to any of claims 1-18, the nucleotide sequence according to claim 19, the expression vector according to claim 20, the host cell according to claim 21, the antibody according to any of claims 22-24, or the immunotherapeutic cellular composition according to any of claims 25-26.

28. The pharmaceutical composition according to the preceding claim further comprising a pharmaceutically acceptable vehicle.

29. The pharmaceutical composition according to any of claims 27-28, where said composition is a vaccine optionally comprising an adjuvant.

30. A combined preparation comprising sequences A and B as they are described in any of claims 1-18.

31. Use of the composition according to any of claims 1-18, the nucleotide sequence according to claim 19, the expression vector according to claim 20, the host cell according to claim 21, the antibody according to any of claims 22-24, or the immunotherapeutic cellular composition according to any of claims 25-26, in the preparation of a medicinal product.

32. Use of the composition according to any of claims 1-18, the nucleotide sequence according to claim 19, the expression vector according to claim 20, the host cell according to claim 21, the antibody according to any of claims 22-24, or the immunotherapeutic cellular composition according to any of claims 25-26, in the preparation of a medicinal product, where the medicinal product is a vaccine.

33. Use of the composition according to any of claims 1-18, the nucleotide sequence according to claim 19, the expression vector according to claim 20, the host cell according to claim 21, the antibody according to any of claims 22-24, or the immunotherapeutic cellular composition according to any of claims 25-26, in the preparation of a medicinal product for the treatment or prevention of aggregating protein-related diseases or disorders.

34. Use of the combined preparation according to claim 30 comprising sequence A and sequence B in the preparation of a medicinal product for combined, simultaneous or sequential administration, for the treatment or prevention of aggregating protein-related diseases or disorders.

35. Use according to any of claims 33-34, where the aggregating protein is α-synuclein and the chaperone is selected from any of those defined in claims 4-14.

36. Use according to any of claims 33-35, where the aggregating protein-related disease or disorder is selected from the list consisting of: Parkinson's disease (PD), Lewy body dementia (LBD), the Lewy body variant of Alzheimer's disease, multiple system atrophy (MSA), Alzheimer's disease, pure autonomic failure (PAF), Down syndrome, amyotrophic lateral sclerosis (ALS), frontotemporal dementia, Gaucher disease, Huntington's disease, type II diabetes, prion disease, Creutzfeldt-Jakob disease, multiple sclerosis, Gerstmann-Sträussler-Scheinker syndrome, Kuru, fatal familial insomnia, cerebrovascular amyloidosis, glaucoma, age-related macular degeneration, neurodegeneration due to age-related protein aggregation, psychiatric syndromes, schizophrenia and/or schizophrenia-like disorders.

37. Use of the composition according to any of claims 1-18, the nucleotide sequence according to claim 19, the expression vector according to claim 20, the host cell according to claim 21, the antibody according to any of claims 22-24, or the immunotherapeutic cellular composition according to any of claims 25-26, where sequence A is α-synuclein or any of the biologically active variants and fragments thereof, sequence B is the chaperone as defined in any of claims 4-14, and the disease is selected from the list consisting of: Parkinson's disease (PD), Lewy body dementia, multiple system atrophy (MSA), Gaucher disease, pure autonomic failure (PAF), and Alzheimer's disease.

38. Use of the composition according to any of claims 1-18, the nucleotide sequence according to claim 19, the expression vector according to claim 20, the host cell according to claim 21, the antibody according to any of claims 22-24, or the immunotherapeutic cellular composition according to any of claims 25-26, where sequence A is α-synuclein or any of the biologically active variants and fragments thereof, sequence B is the chaperone Grp94 or any of the biologically active variants and fragments thereof, in the preparation of a medicinal product for the treatment or prevention of Parkinson's disease.

39. Use according to any of claims 33-34, where the aggregating protein is SOD1 and the chaperone is selected from any of those defined in claims 4-14, preferably the chaperone is Hsp70.

40. Use according to claim 39, where the SOD1-related disease or disorder is ALS.
